(19) United States
(12) Patent Application Publication
(10) Pub. No.: US 2010/0145057 A1
(43) Pub. Date: Jun. 10, 2010

(54) NOVEL PRODRUGS

(75) Inventors: Rajamannar Thennati, Baroda (IN); Biswajit Samanta, Baroda (IN); Ranjan Kumar Pal, Baroda (IN); Srinivasu Kilaru, Baroda (IN); Jignesh Jivani, Baroda (IN); Savajibhai Anil Kumbhani, Baroda (IN); Jay Prakashchandra Adhyapak, Baroda (IN)

Correspondence Address: SCHWEGMAN, LUNDBERG & WOESSNER, P.A., P.O. BOX 2938, MINNEAPOLIS, MN, 55402, US

(73) Assignee: SUN PHARMA ADVANCED RESEARCH COMPANY LTD., ANDHERI(E) MUMBAI (IN)

(21) Appl. No.: 12/531,387

(22) PCT Filed: Mar. 14, 2008
(86) PCT No.: PCT/IN08/00147

§ 371 Date: Feb. 25, 2010

(30) Foreign Application Priority Data
Mar. 15, 2007 (IN) 494/MUM/2007
Mar. 12, 2008 (IN) 510/MUM/2008

(51) Int. Cl. C07C 271/06; C07D 211/34; C07D 401/12

(52) U.S. Cl. 546/227; 560/115; 546/273.7

(57) ABSTRACT

The present invention describes novel prodrugs of formula (I) or their salts, process of preparation and uses thereof.

### FIELD OF INVENTION

The present invention is directed towards novel prodrug of drugs, to their preparation and to uses thereof.

### BACKGROUND OF THE INVENTION

Many of the biologically potent and pharmacologically active drugs fail in the development phase due to lack of structural features needed to cross biological barriers. In other instances the drug molecule may be unstable to the physiological pH conditions in the biological systems. Hence, improving bioavailability is an important criterion for a potential candidate having hurdles in absorption process. Consequently, solutes must possess the optimal physicochemical characteristics for example size, charge, lipophilicity, conformation, hydrogen bonding etc.

In recent years there is an increasing trend in introduction of new drugs as prodrugs. Prodrugs are chemical derivatives of a biologically-active compound which, upon administration, liberates the biologically active compound in vivo. Preparation of prodrug of a drug allows for modification of physicochemical properties of the drug that has an effect of altering pharmacokinetics of the drug for example a prodrug may modify transportation, distribution, metabolism or solubility of the drug in the biological fluids.

Herein, we report a novel prodrug strategy to incorporate functionalities to have the structural features that would modify the topological features like size, charge, conformation and hydrogen bonding characteristics in a drug molecule.

### DESCRIPTION OF THE INVENTION

The present invention relates to novel prodrug compounds of formula-I or salts thereof,

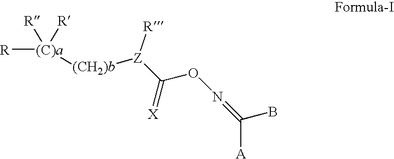

wherein the groups R, R′, R″ and R′″ are independently selected from hydrogen, linear, branched or cyclic alkyl, alkylaryl, aralkyl, aryl, heterocyclic ring; wherein the alkyl group is saturated or unsaturated, and is unsubstituted or substituted with 1 to 5 groups selected from hydroxy, cyano, oxo, carboxylic acid and their derivatives; wherein the aryl and heterocyclic ring is unsubstituted or substituted with 1 to 5 groups selected from alkyl, alkoxy, halo, perhaloalkyl, perhaloalkoxy, haloalkoxy, hydroxy, oxo, cyano, carboxy, acyl, —NR_{P}R_{Q}, wherein R_{P} and R_{Q} are independently selected from hydrogen, alkyl, arylalkyl, alkylaryl, cyclic or heterocyclic ring, —CONR_{M}R_{N}, wherein R_{M} and R_{N} are independently selected from hydrogen, alkyl, aryl, wherein the aryl group is unsubstituted or substituted with alkyl groups;
or any two of R, R′, R″ or R′″ are joined together to form a cyclic moiety which is unsubstituted or substituted with alkyl, perhaloalkyl, alkoxy, halogen, amino, alkylamino, dialkylamino, cyano, carboxy, alkoxycarbonyl, alkanoyl or a group L, wherein L is a compound of formula-P

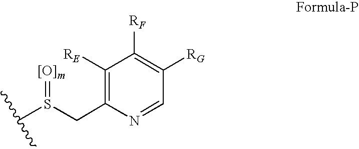

wherein m is 0 or 1; R_{E}, R_{F} and R_{G} are selected from one of the following groups:
i) R_{F} represents a C₁ to C₃ alkoxy group, one of the groups R_{E} and R_{G} represents a C₁ to C₃ alkoxy group and the other represents a hydrogen atom and a C₁ to C₃ alkyl radical or
ii) R_{E} and R_{G} represents hydrogen or methyl; R_{F} represents a group of the formula OCH₂R_{I}, wherein R₁ represents a fluorinated alkyl radical or
iii) R_{E} and R_{G} are independently represent hydrogen, methyl, methoxy, ethoxy, methoxyethoxy or ethoxyethoxy; and R_{F} is selected from methoxy, ethoxy, methoxyethoxy or ethoxyethoxy or
iv) R_{G} is hydrogen, R_{E} represents methyl, and R_{F} represents methoxy substituted by —O-n-propyl;
Z is an atom selected from N, O or S;
X is an atom selected from O or S;
A is selected from hydrogen, C₁ to C₁₀ linear, branched or cyclic alkyl, aryl or heterocyclic ring, wherein the alkyl group is completely saturated or contain unsaturation and is either unsubstituted or substituted, wherein the substitutions are selected from hydroxy, halogen, cyano, carboxy, acyl and derivatives thereof, wherein the aryl and the heterocyclic ring is unsubstituted or substituted with 1 to 5 groups selected from alkyl, alkoxy, halo, perhaloalkyl, perhaloalkoxy, haloalkoxy, hydroxy, cyano, amino, monoalkylamino or dialkylamino groups;
B is selected from hydrogen, cyano, C₁ to C₁₀ alkyl, a group of the formula —COORa, wherein Ra is selected from hydrogen, C₁₋₁₀alkyl, aryl or heteroaryl moiety; or a group of the formula —CONRₓR_{y}, wherein Rₓ and R_{y} are independently selected from hydrogen, C1 to C7 linear, branched or cyclic alkyl, aryl or heterocyclic ring, wherein the alkyl group is completely saturated or contains unsaturation and is either unsubstituted or substituted, wherein the substitutions are selected from hydroxy, halogen, cyano, carboxy, acyl;
or B is a group of formula-II

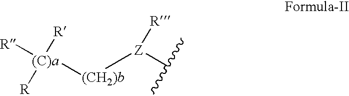

wherein R, R′, R″, R′″, Z have the meanings as defined above;
a is an integer selected from 0 or 1
b is an integer selected from 0 or 1
with a proviso that,
i) when a is 0, b is 0; R′ & R″ are absent and R is directly attached to Z;
ii) when Z is an atom selected from O or S; R′″ is absent;
iii) the compound of formula-I is converted to a compound of formula-III,

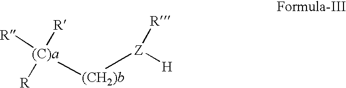

wherein R, R′, R″, R′″, a, b and Z are as defined above.
iv) the compound of formula-III is a biologically active molecule or a diagnostic agent.

The prodrug compound of formula-I may be employed to obtain a compound of formula-III in vivo.

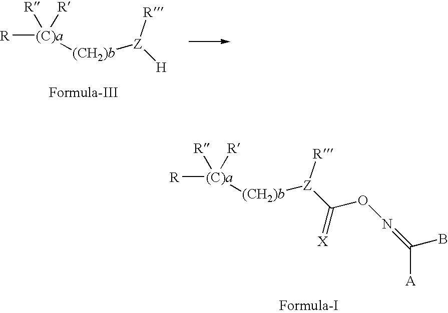

The compound of formula-III, for which the improvement in properties is sought, may be a biologically active molecule or an diagnostic agent which are collectively referred to herein as drug molecule, The compounds of formula I of the present invention thus incorporate changes in structural features of a drug molecule with one or more features/functionalities like, for example, amino group which may be an aliphatic amino or an cyclic amino group, acidic group, alcoholic group, phenolic group, thio group, phosphonates or a combination thereof. The drug molecule may possess suitable chemical groups to enable preparation of the novel prodrugs of formula-I. For example, a suitable drug molecule may be an amino acid, or a drug with amino, phenolic or hydroxyl group, a protein or a peptide, an azole like imidazole, pyrazole, benzimidazole etc. The drug molecule of formula III of the chemical class as mentioned herein above may belong to any of the diverse therapeutic classes, as relevant thereto, for example the drug molecule may be the one acting on the central nervous system for eg a CNS stimulant like phentermine, methylphenidate or an anticonvulsant drug like gabapentin, pregabalin, antispasmodic agent like baclofen, antidepressants like sertraline, antipsychotic like ziprasidone or the drug may be an anticoagulant like tranexamic acid, an antineoplastic agent, a drug acting on the cardiovascular system for example an ACE inhibitor or a beta agonist or antagonists, an antibiotic like β-lactams, macrolides, quinolones, aminoglycosides, morphine or codeine derivative used for relieving pain or an anti-inflammatory drug, antiulcerative drugs like proton pump inhibitors etc. Further the prodrug compounds of formula I may also be useful in improving the solubility of a poorly soluble drug like raloxifene, sertraline, ziprasidone etc. It is to be understood that the examples of the drug molecules, as mentioned hereinabove, are for illustrative purposes and do not limit the scope of the invention.

In one of the embodiments of the present invention, the compound of formula-I is represented by a compound of formula-IV,

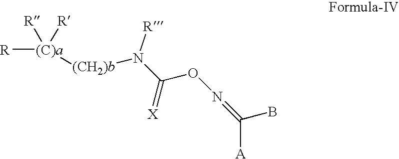

wherein, at least one of the groups R, R′ or R″ contains a carboxylic moiety and the other R, R′ or R″ groups have the meaning as defined for formula-I above, i.e. to say the compound of formula-IV is prodrug of an amino acid.

In another embodiment of the invention, the compound of formula-I is represented by a compound of formula-V,

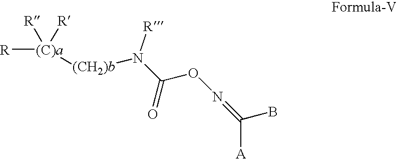

wherein, at least one of the groups viz R, R′ or R″ contains a carboxylic moiety.

The compounds of formula-V, with oximinocarbamate masking charge characteristics and with the appropriate size and hydrogen bonding features exhibit improvement in the pharmacokinetic profile of the parent drug molecule by improving bioavailabilty or increasing half life, as compared to the parent drug. The prodrugs may also be useful in improving the aqueous solubility of the drug, thereby overcoming the problems associated with formulation of the drug in a suitable dosage form.

In another embodiment, the compound of formula-I is represented by a compound of formula-Ia

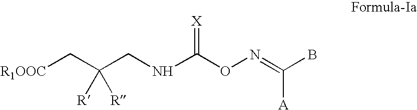

wherein R′ and R″ are connected together with the carbon atom to which they are attached to form a 4, 5 or 6-membered cyclic ring, R₁ is selected from hydrogen atom or a C₁-C₈ alkyl radical, X, A and B have the meanings as defined for compounds of formula-I above. The compounds of formula-Ia are prodrugs of compounds disclosed in U.S. Pat. No. 4,024,175 (referred to as '175 hereinafter), which is incorporated herein as a reference. The compounds of formula-Ia are useful in treatment of certain types of epilepsy, faintness attacks, hypokinesia and cranial traumas. The compounds of formula-Ia may also be useful in treatment of diabetic neuropathic pain,

One of the preferred compounds of the '175 patent is 1-(aminomethyl)cyclohexaneacetic acid, commonly known as Gabapentin (NEURONTIN®, Pfizer), which is approved in United states for the management of postherpetic neuralgia and as an adjunctive therapy in the treatment of partial seizures, The currently approved dosage regimen of Gabapentin typically requires oral administration of 900 mg/day to 4800 mg/day in three divided doses of 300-600 mg each. At the approved dosage range of 900 mg/day to 1800 mg/day, the oral bioavailability is approximately 60-27% respectively. Thus the oral bioavailabilty of gabapentin is low and is non-dose proportional. Thus there is a need for an improved product profile that increases bioavailability, thus eliminating the large doses of the administered dose and improving the side effect profile of gabapentin and other compounds disclosed in the '175 patent. The prodrug compounds of formula-Ia of the present invention possess groups which mask the charge characterstics of the amino group such that a large proportion of the drug remains unionized in the gastrointestinal tract wherein maximal drug absorption occurs. Furthermore, drug absorption occurs without any dose limitation, thereby improving bioavailability of the parent drug.

Another significant problem with many GABA analogs disclosed in the '175 patent, is the intramolecular reaction of the gamma amino group with the carboxyl functionality to form gamma lactam. Formation of gamma lactam presents serious difficulties in formulating gabapentin because of its toxicity (LD50, mouse of >8000 mg/kg for gabapentin, LD50, mouse of 300 mg/kg for the corresponding lactam). Thus, the formation of lactam impurity during synthesis of GABA analogs and/or formulation and/or storage of GABA analogs or compositions of GABA analogs must be minimized for safety reasons. Further, the attempts to prevent lactam formation have not been entirely successful in either synthesis or storage of GABA analogs such as gabapentin or compositions thereof. The prodrugs of formula-Ia of the present invention, presents compounds wherein the amino group is substituted, and is no longer free to undergo spontaneous lactamisation, thus reducing the possibility of formation of lactam impurity during formulation and storage.

In yet another embodiment of the present invention, the compound of formula-I is represented by a compound of formula-Ib or salts thereof

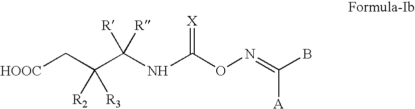

wherein R′ is Hydrogen, R₂ is a straight or branched alkyl of from 1 to 6 carbons, phenyl or cycloalkyl having from 3 to 6 carbon atoms; R″ and R₃ are independently selected from hydrogen or methyl; X, A and B have the meanings as defined for compounds of formula-I above. The compounds of formula-Ib are prodrugs of compounds disclosed in U.S. Pat. No. 6,197,819, which is incorporated herein as a reference. The compounds of formula-Ib are useful in suppression of seizures resulting from epilepsy, the treatment of cerebral ischemia, Parkinson's disease, Huntington's disease and spasticity and also possibly for antidepressant, anxiolytic, and antipsychotic effects. One of the preferred compounds of the '819 patent is (S)-3-(aminomethyl)-5-methylhexanoic acid, which is commonly known as Pregabalin. Pregabalin (LYRICA®) is approved in United States for the treatment of neuropathic pain associated with diabetic peripheral neuropathy, management of postherpetic neuralgia and as an adjunctive therapy in the treatment of partial seizures. The drug has a rapid systemic clearance and thus requires frequent dosing to maintain a therapeutic or prophylactic concentration in the systemic circulation. The conventional approaches to extend the systemic exposure of drugs with rapid clearance involve the use of formulation or device approaches that provide a slow or sustained release of drug within the intestinal lumen. These approaches are well known in the art and normally require that the drug be well absorbed from the large intestine, where such formulations are most likely to reside while releasing the drug. However, many GABA analogs like pregabalin, owing to their amino acid structure, are ionized in the gastrointestinal tract and are thus not absorbed via the large intestine. Rather, these compounds are typically absorbed in the small intestine by a carrier mediated transport mechanism, which is a saturable process. Thus the sustained release technology could not be applied to many GABA analogs like pregabalin. The prodrug compounds of formula-Ib, prevent the ionization of the amino group of pregabalin and other GABA analogs disclosed in the '819 patent, thus the drug is available in non-ionised form, in which form it is absorbed from the large intestine.

Also, like the GABA analogs disclosed in the '175 patent, the GABA analogs disclosed in the '819 patent are also susceptible to spontaneous lactamisation, which occurs due to the intramolecular reaction of the free amino group with the carboxyl functionality. The compounds of formula-Ib of the present invention prevent this intramolecular reaction and thus provide stable GABA analogs.

In a still another embodiment of the present invention, the compound of formula I is represented by a compound of formula-Ic

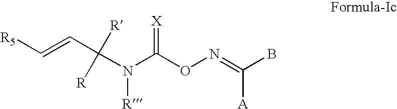

R′ and R′″ is hydrogen;
R₅ is selected from hydrogen or chlorine atom;
R is a group of the formula
—CH═CH—COR₆ or [CH(R₇)]ₙ—COR₆, wherein R₆ is selected from hydroxy, a straight or branched alkoxy group of from 1 to 8 carbon atoms, a lower alkylamino group; R₇ is selected from hydrogen, C₁ to C₄ alkyl, phenyl and substituted phenyl wherein the substituents on phenyl are selected from halogen, C₁ to C₄ alkoxy of from 1 to 4 carbon atoms, and C₁ to C₄ alkyl; n is an integer of from 1 to 5; X, A and B have the meanings as defined for compounds of formula-I above. The compounds of formula-Ic are prodrugs of the compounds disclosed in U.S. Pat. No. 3,960,927, which is incorporated herein as a reference. The compounds of formula-Ic are useful as sedatives. Further, the compounds of formula-Ic wherein R is a group of the formula —CH═CH—COR₆ or —[CH(R₇)]ₙ—COR₆, wherein R₇ is hydrogen and n is an integer from 1 to 5, irreversibly inhibit gamma amino butyric acid transaminase and thereby significantly increase the brain level of GABA. Thus, these compounds are useful in mammals for the treatment of diseased states wherein there is disturabance of the excitation-inhibition interplay as a result of alterations in the level of the GABA and glutamic acid, such as Huntington's chorea, parkinsonism, schizophrenia, epilepsy, depression, hyperkinesis and manic depression disorders. Like the GABA analogs disclosed in the '175 and '819 patents above, the GAba analog disclosed in the '927 patent possess free amino group which can undergo lactamisatrion, yielding lactam impurities during preparation of the bulk drug, formulation as well as during storage. The compounds of formula-Ic provide stable GABA analogs.

In yet another embodiment of the present invention, the compound of formula-I is represented by compounds of formula-Id

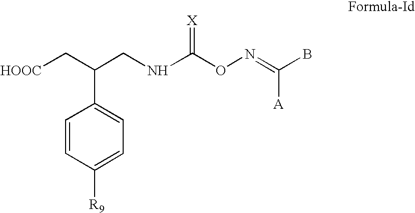

wherein R₉ is selected from a chlorine, bromine, iodine, —CF₃; X, A and B have the meanings as defined for the compounds of formula-I above. The compound of formula Id are prodrugs of compounds disclosed in U.S. Pat. No. 3,471,548 (referred to as '548 hereinafter), which is incorporated herein as a reference. The compounds of formula-Id possess property of inhibiting the activity of neurons involved in motor control. The compounds are thus useful for the alleviation of signs and symptoms of spasticity resulting from multiple sclerosis. Further the compounds of formula-Id may also be useful as an antitussive agent, as an agent for the treatment of angina pectoris, for the treatment of alcohol withdrawal syndrome and promotion of abstinence in alcoholics, for treatment of gastro-esophageal reflux disease, and in the treatment of emesis.

One of the preferred compounds of the '548 patent is a compound of formula-Id, wherein R₉ is chlorine, a compound more commonly known as baclofen. Baclofen is approved in United States and is marketed under the trade name, KEMSTRO®, by Schwarz Pharma. The physicochemical properties of baclofen pose problems in drug formulation and absorption. Being zwitterionic in nature, it can have a net negative, net positive or neutral charge, depending on the pH of the solution. The absorption of baclofen is site specific, in that it is primarily absorbed from the upper small intestine, where it is transported by an amino-acid carrier mediated mechanism. The permeability in the lower intestine is very poor. Further, owing to the structure of baclofen and other compounds of the '548 patent, aqueous solubility is low, which presents problems for dosage formulation. The prodrugs of formula-Id possess a substituted amino group, such that it is not ionized in the gastrointestinal tracts and is present in non-ionised form ready for drug absorption. Also, because of the presence of hydrophilic group, the aqueous solubility of the drug is increased which is advantageous for preparing dosage forms especially solution dosage forms.

In a still another embodiment the compound of formula-I is represented by compounds of formula-Ie

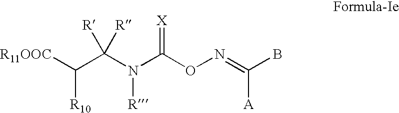

wherein R′ and R′″ are connected to form, together with the N atom to which they are attached a piperidyl ring, R″ is H, R₁₀ is phenyl optionally substituted with C1-4 alkyl; R₁₁ is C₁ to C₄ alkyl; X, A and B have the meanings as defined for compounds of formula-I above. The compounds of the formula-Ie are prodrugs of compounds disclosed in U.S. Pat. No. 2,507,631, which is incorporated herein as a reference. The compounds of formula-Ie are CNS stimulants and are useful in the treatment of Attention deficit hyperactivity disorders (ADHD). The compounds of formula-Ie may also be useful in treatment of cognitive decline in patients with AIDS or AIDS-related conditions

In another embodiment the compounds of formula-I is represented by compounds of formula-If

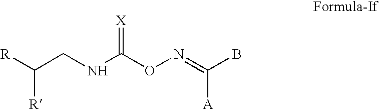

wherein R and R′ are connected together to form a 6-membered saturated cyclic ring which is substituted by COOH; X, A and B have the meanings as defined for compounds of formula-I above. The compounds of formula-If are prodrugs of the compound disclosed in U.S. Pat. No. 3,950,405 (referred to as '405 hereinafter), which is incorporated herein as a reference. The compounds of formula-If are useful in the treatment of disorders wherein the plasmin activity in blood is very high, for example the compounds may be useful in patients with hemophilia to reduce or prevent hemorrhage or to reduce the need for replacement therapy during and following tooth extraction. One of the preferred compound of the '405 patent, the trans isomer of the compound of formula-IX, i.e. trans-4-(aminomethyl)cyclohexanecarboxylic acid, commonly known as Tranexamic acid, is approved in United States and is marketed under the brand name CYKLOKAPRON® by Pharmacia and Upjohn in patients with hemophilia for short term use (2-8 days) to reduce or prevent hemorrhage and reduce the need for replacement therapy during and following tooth extraction. Tranexamic acid exhibits poor oral bioavailability in that only 35-40% of the orally administered dose is absorbed. Consequently, a fairly high dosages of the drug is prescribed, typically about 3 gm to about 6 gms in 24 hours. Such large intake causes gastrointestinal side effects in patients, which may be due to local irritation caused due to unabsorbed drug. Improvement in extent absorption of these drugs may lead to administration of lower dosages and resultant improvement in the side effect profile of the drug. The prodrugs of formula-If above, decrease the basicity of the free amino group due to substitution and thus enhance drug absorption via gastrointestinal tract thereby improving the bioavailability of these drugs.

In other embodiment compounds of formula I is represented by compounds of formula-Ig

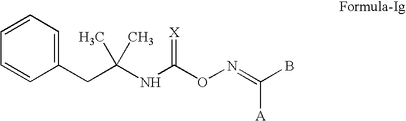

wherein the substitutents X, B and A have the meanings as defined above. The compounds of Formula-Ig are prodrugs of compounds disclosed in U.S. Pat. No. 2,408,345, which is incorporated herein as a reference. The compounds of formula-Ig are useful as CNS stimulants and as appetite suppressants.

In yet another embodiment of the present invention, the compounds of formula-I are represented by compounds of formula-Ih

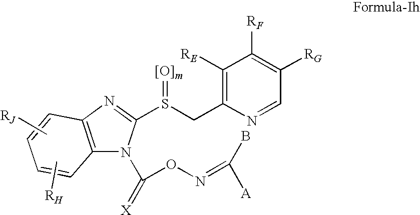

wherein m is 0 or 1; the groups R_{J}, R_{H}, R_{E}, R_{F} and R_{G} are selected from one of the following groups:
i) R_{F} represents a C₁ to C₃ alkoxy group, one of the groups R_{E} and R_{G} represents a C₁ to C₃ alkoxy group and the other represents a hydrogen atom and a C₁ to C₃ alkyl radical R_{J} is at 6-position and represents hydrogen, halo, trifluoromethyl, a C₁ to C₃ alkyl radical or a C₁ to C₃ alkoxy radical which is optionally, predominantly or completely substituted by fluorine atoms, R_{H} is at 5-position and represents a C₁ to C₃ alkoxy radical which is optionally, predominantly or completely substituted by fluorine atoms or a chlorodifluormethyl radical;
ii) R_{E} and R_{G} represents hydrogen or methyl; R_{F} represents a group of the formula OCH₂R_{I}, wherein R_{I} represents a fluorinated alkyl radical, Rⱼ is hydrogen, R_{H} is selected from hydrogen, methoxy or trifluoromethyl;
iii) R_{E} and R_{G} are independently represent hydrogen, methyl, methoxy, ethoxy, methoxyethoxy or ethoxyethoxy; and R_{F} is selected from methoxy, ethoxy, methoxyethoxy or ethoxyethoxy; Rⱼ and R_{H} are independently selected from the group consisting of hydrogen, alkyl, halogen, carbomethoxy, carboethoxy, alkoxy, and alkanoyl;
iv) R_{G} is hydrogen, R_{E} represents methyl, and R_{F} represents methoxy substituted by —O-n-propyl, Rⱼ and R_{H} are independently selected from the group consisting of hydrogen, halogen, C₁ to C₆ alkyl, halogenated C₁ to C₆ alkyl, C₁ to C₆ alkoxy, C₁ to C₆ alkoxycarbonyl or carboxyl group. The compounds of formula-Ih are prodrugs of compounds disclosed in U.S. Pat. Nos. 4,758,579, 4,508,905, 5,045,552 and European Patent No. 174726. The compounds disclosed in the patent references mentioned above, can be generalized as those containing pyridylsulfinylbenzimidazole core structure. These compounds inhibit the gastric (H⁺, K⁺)-ATPase and are commonly referred to as Proton pump inhibitors (PPI) or “prazoles”. For the past two decades, the stability of the PPIs has been of the concern for many scientists and there have been several attempts to increase the stability of PPIs. It has been shown that, the prazoles undergo acid activation to generate the reactive species which bind to the active site of ATPase. Further, it has been suggested that while protonation of the pyridine moiety (or the pka1 of pyridine nitrogen) determines the selective accumulation of the prazoles at the active site, it is the protonation of benzimidazole moiety (or pka2 of the benzimidazole nitrogen) which plays a decisive role in the activation of these compounds to the reactive species and thus determines the relative stability of these PPIs (Shin J. M., Cho, Y. M., Sachs G., Journal of American Chemical Society, 2004, 126, 7800-7811) In the compounds of formula-Ih, the benzimidazole nitrogen, which is substituted, has a reduced ability to undergo protonation thereby chemical degradation, in the gastrointestinal fluids or when stored. The compounds of formula-Ih are useful in inhibiting gastric acid secretion and thus are useful to prevent ulcer formation. These compounds may be useful in conditions such as duodenal ulcers, gastroesophageal reflux disease, erosive esophagitis, hypersecretory conditions as Zollinger-Ellison syndrome.

In a more preferred embodiment, the compounds of formula-I is represented by compounds of formula-Ii

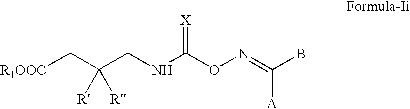

wherein R′ and R″ are connected together with the carbon atom to which they are attached to form a 4, 5 or 6-membered saturated cyclic ring, R₁ is selected from hydrogen atom or a C₁-C₈ alkyl radical;
B is a group of the formula-VIII

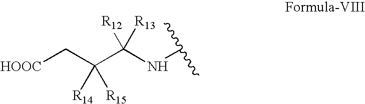

wherein R₁₂ is hydrogen, R₁₃ and R₁₅ are independently selected from hydrogen or methyl, R₁₄ is a straight or branched alkyl of from 1 to 6 carbons, phenyl or cycloalkyl having from 3 to 6 carbon atoms; X and A have the meanings as defined for compounds of formula-Ib above. The compounds of formula-Ii are prodrugs, which are converted in-vivo, by chemical or enzymatic hydrolysis to compounds of formula-M and Formula-N, which are the compounds disclosed in '175 and '819 patents, which are incorporated herein as a reference.

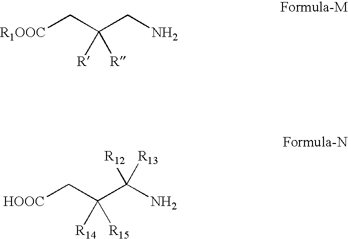

The compounds of formula-Ii are useful in treatment of certain types of epilepsy, faintness attacks, hypokinesia and cranial traumas. The compounds of formula-Ii may also be useful in treatment of diabetic neuropathic pain.

In a more preferred embodiment, compounds of formula-Ia is represented by a compounds of formula Ij

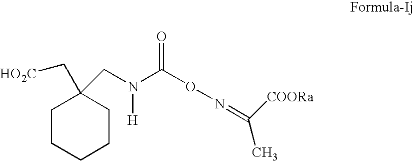

wherein Ra has the meaning as defined in formula-Ia above.

In another more preferred embodiment, compounds of formula-Id is represented by a compound of formula-Ik

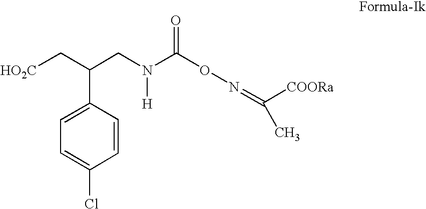

wherein Ra has the meaning as defined in formula-Id above.

The following are definitions of terms used in this specification.

As used herein the term ‘alkyl’ refers to a linear, branched, or cyclic hydrocarbon moiety optionally containing one or more unsaturations. The term includes in its definition radicals such as linear alkyl substituted with cycloalkyl or vice versa. As used herein, alkyl including unsaturations is to be understood as meaning ‘alkenyl’ and/or ‘alkynyl’. Exemplary alkyl groups include methyl, ethyl, n-propyl, i-propyl, n-butyl, t-butyl, n-pentyl, 3-pentyl, 2-octyl and the like. Exemplary alkenyl groups include ethenyl, propenyl, 1-butenyl, (Z)-2-butenyl, (E)-3-methylbut-2-enyl, (E)-2,4-pentadienyl, (Z)-3-heptenyl and the like. Exemplary alkynyl groups include ethynyl, propynyl, 1-butynyl, 2-butynyl, 4-methyl-2-pentynyl, 2,4-hexadiynyl and the like.

The term “alkoxy” as used herein refers to an alkyl group, as defined herein, appended to the parent molecular moiety through an oxygen atom. Representative examples of alkoxy include, but are not limited to, methoxy, ethoxy, propoxy, isopropoxy, butoxy, tert-butoxy, pentyloxy, hexyloxy.

As used herein ‘aryl’ is to be understood as meaning aromatic ring system which may be monocyclic or polycyclic. The aryl ring may be fused with a cyclic or a heterocyclic ring. Example of aryl group includes phenyl, naphthyl, anthracenyl, phenanthryl. etc.

As used herein the term “alkylaryl” refers to the group Rₛ—Rₜ, wherein Rₛ is an aryl group as defined hereinabove substituted by Rₜ, an alkyl group defined above.

As used herein the term “aralkyl” refers to a group —Rᵤ—Rᵥ, wherein Rᵤ is an alkyl group as defined hereinabove substituted by an aryl group as defined above.

As used herein ‘heterocyclyl’ or ‘heterocyclic ring’ is to be understood as meaning monocyclic or polycyclic ring systems which, in addition to carbon, also contain one or more hetero atoms, such as, for example, nitrogen, oxygen or sulfur which may be unsaturated or wholly or partly saturated. This definition furthermore includes ring systems in which the heterocyclyl rings are aromatic, i.e. ‘heteroaryl’, or heterocyclic radical that is fused with benzene rings.

As used herein the term “cyclic moiety” refers to monocyclic or bicyclic aliphatic hydrocarbon radical containing 4-7 carbon atoms or heterocyclic moiety as defined hereinabove. The cyclic moiety may be fully saturated or may contain unsaturations therein.

The phrase “carboxylic acid and their derivatives” as used herein refers to the amide, ester derivatives of carboxylic acid, sulfonic acid, sulfonates, phosphoric acid, and phosphonates thereof. The amide derivative of the acid may be a group of the formula —CONR₁₂R₁₃ wherein R₁₂ and R₁₃ are independently selected from hydrogen, alkyl, aryl, wherein the aryl group is unsubstituted or substituted with alkyl groups; the ester derivative of the carboxylic acid may be a group of the formula —COOR_{z}, wherein R_{z} is selected from hydrogen, alkyl, aryl, alkylaryl, aralkyl, cyclic or heterocyclic ring. The sulfonates may be alkyl, aryl, aralkyl or alkylaryl sulfonates, further the phosphonates may be alkyl, aryl aralkyl, alkylaryl phosphonates.

The term “protecting group” refers to a group which, when bound to one or more group(s), limits reactions occurring at these group(s) and which protecting groups can be removed by conventional chemical or enzymatic steps to re-establish the group(s). The particular removable protecting group employed is determined by the nature of the compounds and chemical processes being utilized.

Salts of compounds of formula I may be an acid addition salt or a base addition salt depending on the presence of basic or acidic groups in the compounds. Salts are preferably pharmaceutically acceptable salts. Acid addition salts may be salt of compounds of formula I with basic amino group with an organic or an inorganic acid. Suitable inorganic acids are, for example halogen acids, such as hydrochloric acid, sulfuric acid or phosphoric acid. Suitable organic acids are, for example, carboxylic, phosphonic, sulfonic or sulfamic acids, for example acetic acid, propionic acid, ethane-1,2-disulfonic acid, benzenesulfonic acid, N-cyclohexylsulfamic acid etc.

Basic addition salts may be salts of acidic groups for example carboxylic, sulfonic acid group of compounds of formula I with bases, for example, metal or ammonium salts, such as alkali metal or alkaline earth metal salts, e.g. sodium, potassium, magnesium or calcium salts, or ammonium salts with ammonia or suitable organic amines, such as tertiary monoamines, e.g. triethylamine or tris(2-hydroxyethyl)amine, or heterocyclic bases, e.g. N-ethylpiperidine or N,N′-dimethylpiperazine.

Asymmetric centers can exist in the present compounds and the individual isomers are within the scope of the present invention. The individual stereoisomers of the compounds can be prepared by synthesis from chiral starting materials or by preparation of racemic mixtures and separation by conversion to a mixture of diastereomers followed by separation, chromatographic techniques, or direct separation of the enantiomers on chiral chromatographic columns.

Geometric isomers can exist in the present compounds. The invention contemplates various geometric isomers and mixtures thereof resulting from the disposition of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

The prodrugs of formula I release the drug molecule under physiological conditions, which then elicits its effects. Accordingly, the compounds of formula-I are useful for therapeutic and/or diagnostic purposes.

The novel prodrug compound of formula-I may be administered in the form of a suitable pharmaceutical composition comprising therapeutically effective amount of one or more of the compounds of the invention with one or more therapeutically acceptable excipients. The term “therapeutically acceptable excipient,” as used herein, represents a non-toxic, solid, semisolid or liquid filler, diluent, encapsulating material, or formulation auxiliary of any type. Examples of therapeutically acceptable excipients include sugars; cellulose and derivatives thereof; oils; glycols; solutions; buffering, coloring, releasing, coating, sweetening, flavoring, and perfuming agents; and the like. The compositions may also be administered or co-administered in sustained release dosage forms.

The suitable pharmaceutical compositions may be in the form of solid, liquid or semisolid dosage form and may include for example, tablets, capsules, pills, granules, dragees, powders, suppositories, solution, suspension, emulsion or the like. The composition may be formulated for immediate or sustained release of the active ingredient by the choice of suitable excipients.

The compounds of formula-I may be useful in therapy or for diagnostic purposes where they may be used either atone, or in combination with another drug for an additive or a synergistic effect.

The invention is illustrated but not restricted by the description in the following examples.

| Example | |
|---|---|
| No. | Name |
| 1 | N-[(Oximinopropane-2-yl)carbonyl]-1-aminomethyl cyclohexaneacetic |
| | acid. |
| 2 | N-[(Oximinocyclohexane)carbonyl]-1-aminomethyl cyclohexaneacetic |
| | acid. |
| 3 | N-[(Oximinocyclopentane)carbonyl]-1-aminomethyl cyclohexaneacetic |
| | acid. |
| 4 | N-[(Oximino-1,1-dicyclopropyl methane)carbonyl]-1-aminomethyl |
| | cyclohexaneacetic acid. |
| 5 | N-[(Ethyl oximinopropionate-2-yl)carbonyl]-1-aminomethyl |
| | cyclohexaneacetic acid. |
| 6 | N-[(Methyl oximinopropionate-2-yl)carbonyl]-1-aminomethyl |
| | cyclohexaneacetic acid. |
| 7 | N-[(Cyclopropyl methyl oximinopropionate-2-yl)carbonyl]-1-amino |
| | methylcyclohexaneacetic acid. |
| 8 | N-[(Isopropyl oximinopropionate-2-yl)carbonyl]-1-aminomethyl |
| | cyclohexaneacetic acid. |
| 9 | N-[(n-Butyl oximinopropionate-2-yl)carbonyl]-1-aminomethyl |
| | cyclohexaneacetic acid. |
| 10 | N-[(Isobutyl oximinopropionate-2-yl)carbonyl]-1-aminomethyl |
| | cyclohexaneacetic acid. |
| 11 | N-[(Ethyl-2-{2-aminothiazole}oximinoethanoate-2-yl)carbonyl]-1- |
| | aminomethyl cyclohexaneacetic acid. |
| 12 | N-[(Oximinopropionic acid-{4-amino-3-(2-methylpropyl)butanoic} |
| | amide-2-yl)carbonyl]-1-aminomethyl cyclohexaneacetic acid. |
| 13 | N-[(Oximinopropionic acid dimethyl amide-2-yl)carbonyl]-1- |
| | aminomethyl cyclohexaneacetic acid. |
| 14 | N-[(Oximinopropionic acid pyrrolidine amide-2-yl)carbonyl]-1- |
| | aminomethyl cyclohexaneacetic acid. |
| 15 | N-[(Ethyl oximinopropionate-2-yl)carbonyl]-4-amino-3-(4- |
| | chlorophenyl)butanoic acid. |
| 16 | N-[(Isopropyl oximinopropionate-2-yl)carbonyl]-4-amino-3-(4- |
| | chlorophenyl)butanoic acid. |
| 17 | N-[(Methyl oximinopropionate-2-yl)carbonyl]-4-amino-3-(4- |
| | chlorophenyl)butanoic acid. |
| 18 | N-[(Oximinopropane-2-yl)carbonyl]-4-amino-3-(4-chlorophenyl) |
| | butanoic acid. |
| 19 | (R)-N-[(Methyl oximinopropionate-2-yl)carbonyl]-4-amino-3-(4- |
| | chlorophenyl)butanoic acid. |
| 20 | (R)-N-[(Ethyl oximinopropionate-2-yl)carbonyl]-4-amino-3-(4- |
| | chlorophenyl)butanoic acid. |
| 21 | (R)-N-[(Oximinopropane-2-yl)carbonyl]-4-amino-3-(4-chlorophenyl) |
| | butanoic acid. |
| 22 | (±)-Threo-N-[(Methyl oximinopropionate-2-yl)carbonyl]-1-phenyl-1-(2- |
| | piperidine)aceticacid methyl ester. |
| 23 | (±)-Threo-N-[(Ethyl oximinopropionate-2-yl)carbonyl]-1-phenyl-1-(2- |
| | piperidine)aceticacid methyl ester. |
| 24 | N-[(Ethyl oximinopropionate-2-yl)carbonyl]-1,1-dimethyl-2-phenyl |
| | ethylamine. |
| 25 | (S)-N-[(Ethyl oximinopropionate-2-yl)carbonyl]-4-amino-3-(2- |
| | methylpropyl)butanoic acid. |
| 26 | Trans-N-[(Ethyl oximinopropionate-2-yl)carbonyl]-4-(aminomethyl) |
| | cyclohexanecarboxylic acid. |
| 27 | Trans-N-[(Methyl oximinopropionate-2-yl)carbonyl]-4-(aminomethyl) |
| | cyclohexanecarboxylic acid. |
| 28 | Trans-N-[(Isopropyl oximinopropionate-2-yl)carbonyl]-4-(aminomethyl) |
| | cyclohexanecarboxylic acid. |
| 29 | Trans-N-[(Oximinopropane-2-yl)carbonyl]-4-(aminomethyl)cyclohexane |
| | carboxylic acid. |
| 30 | N-[(Oximinopropane-2-yl)carbonyl]-5-methoxy-2-[[(4-methoxy-3,5- |
| | dimethyl-2-pyridinyl)methyl]sulfinyl]-1H-benzimidazole |
| 31 | N-[(Ethyl oximinopropionate-2-yl)carbonyl]-5-methoxy-2-[[(4-methoxy- |
| | 3,5-dimethyl-2-pyridinyl)methyl]sulfinyl]-1H-benzimidazole |
| 32 | N-[(Oximinopropionic acid dimethyl amide-2-yl)carbonyl]-5-methoxy-2- |
| | [[(4-methoxy-3,5-dimethyl-2-pyridinyl)methyl]sulfinyl]-1H- |
| | benzimidazole |
| 33 | N-[(Oximinopropane-2-yl)carbonyl]-5-(difluoromethoxy)-2-[[(3,4- |
| | dimethoxy-2-pyridinyl)methyl] sulfinyl]-1H-benzimidazole |
| 34 | N-[(Ethyl oximinopropionate-2-yl)carbonyl]-5-(difluoromethoxy)-2- |
| | [[(3,4-dimethoxy-2-pyridinyl)methyl]sulfinyl]-1H-benzimidazole |
| 35 | N-[(Oximinopropionic acid dimethyl amide-2-yl)carbonyl]-5- |
| | (difluoromethoxy)-2-[[(3,4-dimethoxy-2-pyridinyl)methyl]sulfinyl]-1H- |
| | benzimidazole |
| 36 | N-[(Oximinopropane-2-yl)carbonyl]-2-[[[3-methyl-4-(2,2,2- |
| | trifluoroethoxy)-2-pyridinyl]methyl]sulfinyl]-1H-benzimidazole |
| 37 | N-[(Ethyl oximinopropionate-2-yl)carbonyl]-2-[[[3-methyl-4-(2,2,2- |
| | trifluoroethoxy)-2-pyridinyl]methyl]sulfinyl]-1H-benzimidazole |
| 38 | N-[(Oximinopropionic acid dimethyl amide-2-yl)carbonyl]-2-[[[3- |
| | methyl-4-(2,2,2-trifluoroethoxy)-2-pyridinyl]methyl]sulfinyl]-1H- |
| | benzimidazole |
| 39 | N-[(Oximinopropane-2-yl)carbonyl]-2-[[[4-(3-methoxypropoxy)-3- |
| | methyl-2-pyridinyl]methyl]sulfinyl]-1H-benzimidazole |
| 40 | N-[(Ethyl oximinopropionate-2-yl)carbonyl]-2-[[[4-(3- |
| | methoxypropoxy)-3-methyl-2-pyridinyl]methyl]sulfinyl]-1H- |
| | benzimidazole |
| 41 | N-[(Oximinopropionic acid dimethyl amide-2-yl)carbonyl]-2-[[[4-(3- |
| | methoxypropoxy)-3-methyl-2-pyridinyl]methyl]sulfinyl]-1H- |
| | benzimidazole |

### Process of Preparation

The novel prodrugs compounds of formula-I of the present invention can be prepared from commercially readily available compound of formula-III or a salt thereof. The process of preparation can be outlined in the schemes Ito VIII below.

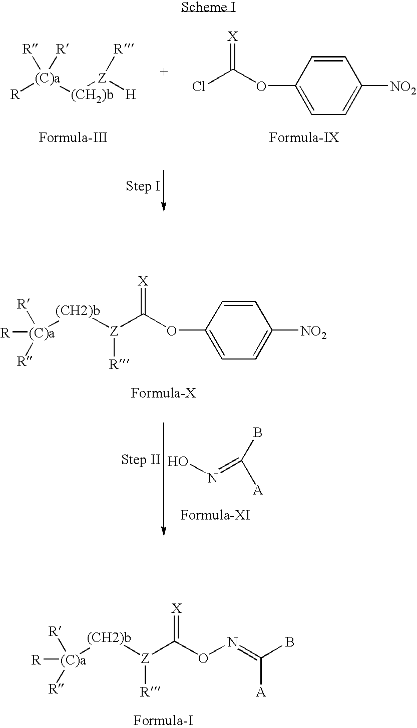

In the reaction scheme I, Step 1, a compound of formula III is treated with a compound of formula IX, wherein X has the meaning as defined for formula I above, to obtain the compound of formula X. The reaction can be carried out in presence of one or more bases and in a suitable solvent. Suitable base for the reaction may be organic or an inorganic base. The inorganic base which may be used for the reaction may be selected from alkali and alkaline metal salts of hydroxide, carbonates, bicarbonates, hydrides etc. for example, sodium hydroxide, potassium hydroxide, or ammonium hydroxide. The organic base which may be used for the reaction is selected from triethylamine, pyridine, picolines, quinoline, N-methylmorpholine etc, preferred being triethylamine, N,N-diisopropylethylamine.

The reaction may be carried out in presence of a solvent or a mixture of solvents. As the solvent, any solvent may be used as long as it does not adversely effect the reaction, and may be, for example, chlorinated solvents like methylenedichloride, ethylene dichloride, ethers such as tetahydrofuran, diethylether, alcohols like methanol, ethanol, isopropyl alcohol, propyl alcohol, including other solvents like acetone, dimethylformamide, dimethylsulfoxide, dioxane, ethyl acetate, toluene, dichloromethane, chloroform or mixed solvents thereof. The suitable temperatures for the reaction may be in the range of 0° C. to about 100° C., preferably the reaction can be carried out in the range of 0° C. to about 50° C.

Further the reaction may be carried out in presence of a base such as inorganic or an organic base. Preferably, the reaction may be carried out in presence of an organic bases such as, but not limited to triethylamine, N-methylmorpholine, pyridine, picolines, quinolines, etc, most preferably in presence of N,N-diisopropylethylamine.

The compound of formula-III is commercially available or, alternatively, may be prepared from the methods known in the art. For compounds of formula III which possess more than one group which may undergo the reaction, the undesirable reaction at the other reacting group may be prevented by use of a suitable protecting group. The protected compound of formula-III may then undergo the reaction as depicted in the schemes below. After the completion of the desired reaction, the protecting group may be removed by a deprotection step. The protecting group which can be used for the said purpose depends on factors such as the functional group to be protected; the reactive species involved in the reaction, the reaction conditions employed, the selection of suitable protecting group for a particular reaction is within the level of a skilled person. For example, the carboxylic acid group may be protected by preparing esters thereof, for example, alkyl esters like methyl ester, t-butyl esters, benzyl esters, silyl esters etc. likewise, the undesired reaction at the hydroxyl functional group can be prevented by using protecting groups like silyl ethers, such as a trimethylsilyl ether, a tert-butyldimethylsilyl ether, or a tert-butyldiphenylsilyl ether, the thiol moiety may be protected by formation of thioester, such as a thioacetate or a thiobenzoate or as a disulfide. Further the protection as well as the deprotection reactions is well known in the art. For example the carboxylic acid group which is protected by preparing alkyl esters thereof, may be deprotected by using an acid or a base, deprotection of benzyl esters may be accomplished by hydrogenolysis. Deprotection of the thiol moiety may be carried out using zinc in dilute aqueous acid, triphenylphosphine in water and sodium borohydride which reduce the disulfide groups while aqueous base or sodium methoxide in methanol may be used hydrolyze thioesters.

Step 2 of the reaction involves reaction of a compound of formula-X with a compound of formula-XI to obtain a compound of formula-I, in presence of a base and a suitable solvent. The base which can be used in the reaction may be an inorganic or an organic base. The inorganic base which may be used for the reaction may be selected from alkali or alkaline metal hydroxides, carbonates or hydrides, for example sodium hydroxide, potassium carbonate, sodium hydride etc. the organic base which may be used for the reaction may be selected from triethylamine, pyridine, picolines, quinoline, N-methylmorpholine, potassium O-tertiarybutoxide, etc, preferred being N,N-diisopropylethylamine, triethylamine.

The solvent which may be used for the reaction may be selected from aromatic hydrocarbon solvents selected from toluene, xylene etc. or polar solvents like acetonitrile, tetrahydrofuran ether, dichloromethane, dichloroethane, methylisobutylketone etc. Preferred being methylisobutyl ketone, tetrahydrofuran.

In an alternative method the compounds of formula-III are converted to their corresponding chloroformates of formula-XII by reaction with phosgene or triphosgene. (Scheme II) The reaction may be carried out in an inert solvent in presence of a base like triethylamine, pyridine. The carbamates of formula XII may further be reacted with an oxime of formula-XI to obtain a compound of formula I.

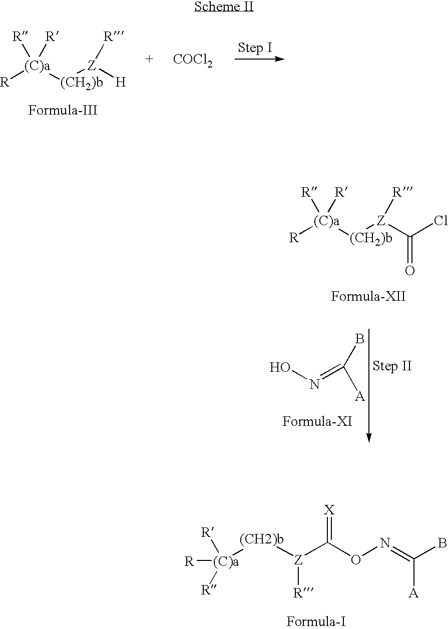

The compounds of formula-I may also be prepared by a process as outlined in Scheme III below, wherein the oxime of formula-XI is treated with 4-nitrophenyl chloroformate or 4-nitrophenylchlorothioformate of formula-IX to obtain the corresponding oximinocarbonyloxy compound of formula-XIII. The compound of formula-XIII can then be reacted with compound of formula-Ill to yield the compounds of formula-I. The reaction may be carried out in presence of an organic or an inorganic base in an organic solvent.

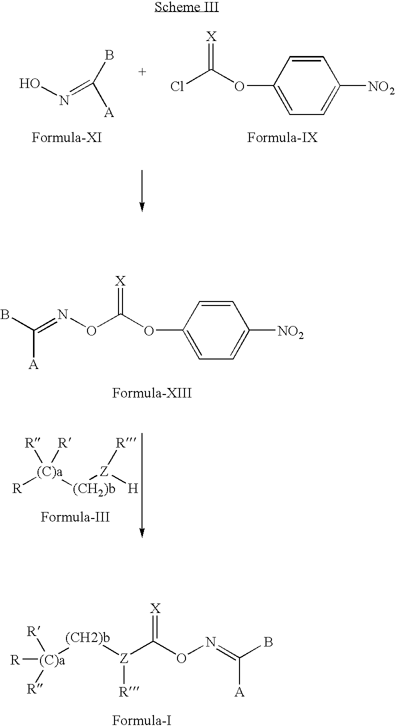

In yet another method of preparing compounds of formula-I, the oxime of formula-XI may be treated with phosgene (COCl₂) or triphosgene to yield the compound of oximino chloroformate formula-XIV, which may be further treated with compounds of formula-III to obtain the compounds of formula-I.

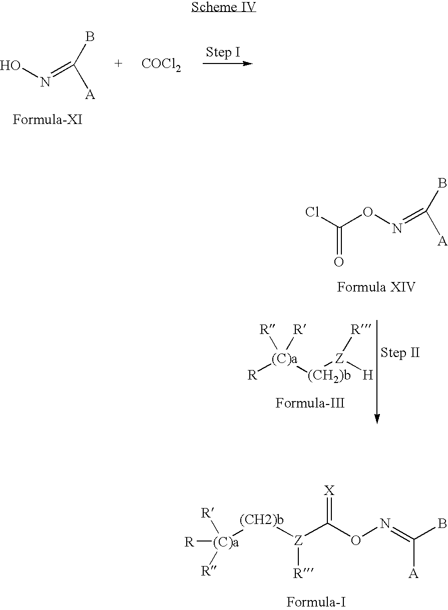

Scheme V to VIII denote the general method of synthesis analogous to Schemes I to IV, for a subclass of compounds of formula-I, wherein R and R′″ form together with the nitrogen atom to which they are attached an aryl or a heteroaryl ring. Scheme V depicts reaction of compounds of formula-XV, wherein R_{J} and R_{H} have the meaning as defined above, D and E are independently selected from a radical of the formula —N—, CH₂— or —CH—, wherein the hydrogen atom(s) may be further replaced with a substitutent L, wherein L is as defined above in formula-I.

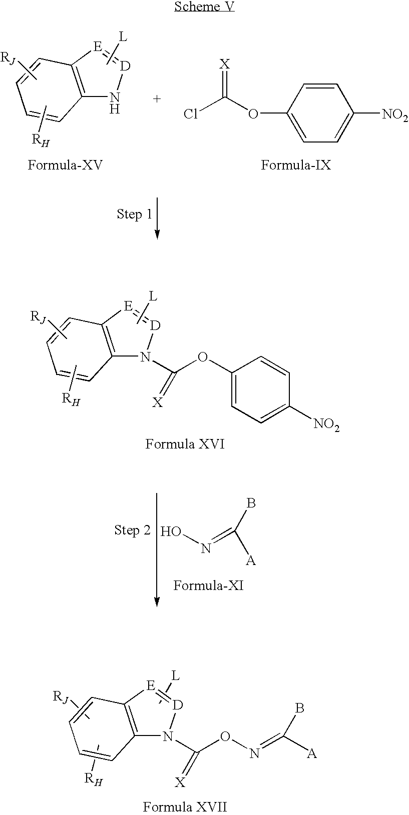

Scheme VI illustrates a process wherein the compound of formula XV is treated with phosgene or triphosgene to obtain the carbamate derivative of formula XVIII. The reaction may be carried out in presence of a base and in a suitable solvent. A suitable base for the reaction may be an inorganic or an organic base. Suitable inorganic bases may be for example, like carbonates, bicarbonates, hydroxides of sodium, potassium, lithium etc. and the suitable organic base may be selected from amines like triethylamine, N,N-diisopropylamine, pyridine, picoline etc.

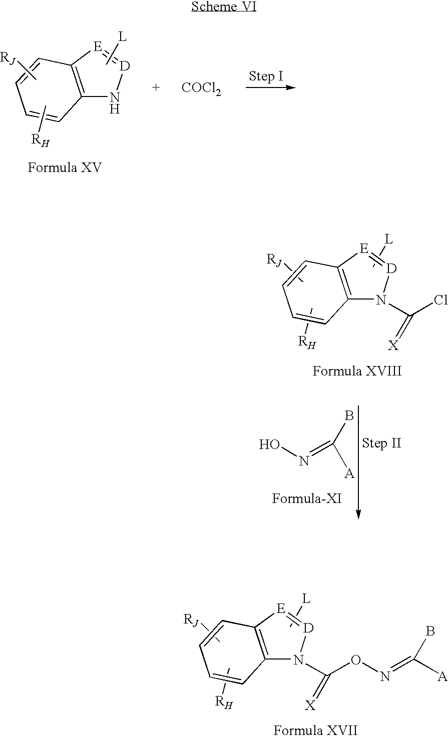

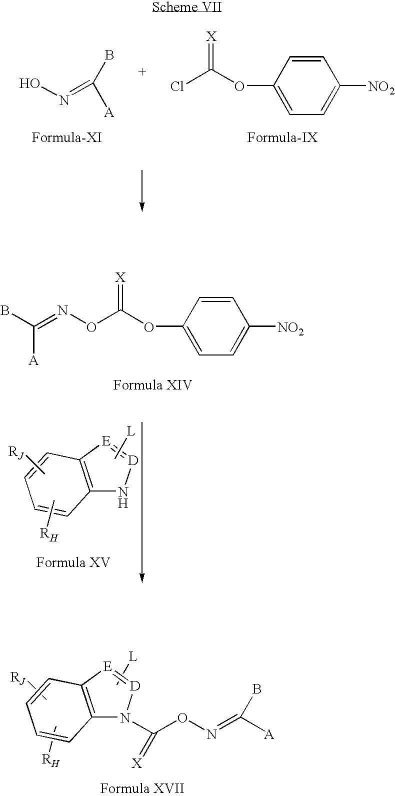

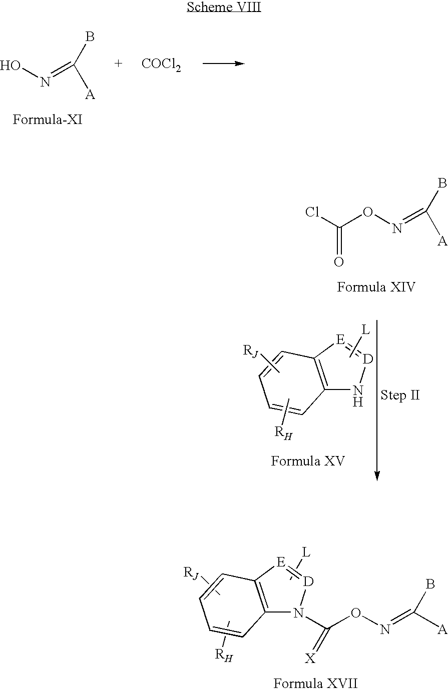

The compounds of formula-XI may be prepared by reacting an aldehyde or a ketone compound of the formula-XIX

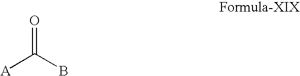

with a salt of hydroxylamine in presence of a base and a solvent. The salt of hydroxylamine may be hydroxylamine hydrochloride, hydroxylamine sulfate, hydroxylamine bisulfate or hydroxylamine phosphate. The base which can be used for the reaction may be an inorganic or an organic base for example, ammonium hydroxide, potassium hydroxide, sodium hydroxide, lithium hydroxide, triethylamine, N,N-disiopropylamine and the like. Preferred being triethylamine. The reaction may be carried out in an aqueous or organic solvent or mixture thereof. Alternatively, the compounds of formula-XIX wherein B is an amide can be obtained by reacting the corresponding carboxylic acid with a required amine. The corresponding hydroxyl group of the carboxylic acid may be first activated using groups such as p-nitrophenyl chloroformate, 1,3-dicyclohexylcarbodiimide, and hydroxybenzotriazide etc. the activated carboxylic acid derivatives can then be treated with the corresponding amine compound to yield the respective amide.

### EXAMPLES

The invention is further illustrated with the preparation of following examples, which may be suitably modified or employed for making various other prodrug derivatives. The method of producing some of the starting compounds used in the examples is described as reference examples.

### Reference Example I

### Preparation of ethyl 2-hydroxyiminopropionate

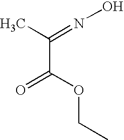

To a solution of 65.9 gm (0.948 mol) of hydroxylamine hydrochloride in 400 ml DM water was added 100 gm of triethylamine at less than 30° C. and stirred for 15 minutes at 25-30° C. To this mixture added a solution of 100 gm (0.861 mol) ethylpyruvate in 100 ml rectified spirit at 25-30° C. over a period of 30 minutes and stirred the reaction mass at 45-50° C. for 1.0 hr. Rectified spirit was distilled at below 50° C. under vacuum and added 200 ml DM water, cool the suspension to 0-5° C. and filtered the solid and washed with chilled DM water, dried at 50-55° C. under vacuum to get ethyl 2-hydroxyiminopropionate.

### Reference Example-II

### Preparation of 2-hydroxyimino propionic acid

### Step-I: Preparation of 2-hydroxyimino propionic acid

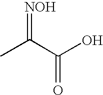

An aqueous solution (390 ml) of 47.6 gm (1.19 mol) of sodium hydroxide was added to a solution of 130.0 gm (0.99 mol) of ethyl-2-hydroxyiminopropionate in ethanol (910 ml) at room temperature. Reaction mixture was heated at 70° C. for 1.5 hr. Reaction mixture was concentrated under vacuum and DM water (500 ml) was added to the residue. Aqueous layer was washed with diethyl ether (2×250 ml) and acidified (pH˜2) with 6N HCl solution. Aqueous layer was saturated with solid sodium chloride and extracted with THF (3×500 ml). Combined THF layer was dried over anhydrous sodium sulphate and distilled under vacuum to get light yellow solid, which was washed with THF (1×720 ml) to furnish 2-hydroxyimino propionic acid

### Step-II: Preparation of 1-pyrrolidin-1-ylpropane-1,2-dione-2-oxime

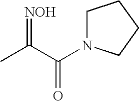

7.86 gm (0.058 mol) of 1-hydroxy benztriazole was added to a stirred solution of 4.0 gm (0.038 mol) of 2-hydroxyimino propionic acid in DMF (40 ml) and stirred for 10 minutes at room temperature. 3.21 ml (0.038 mol) of pyrrolidine followed by 8.93 gm (0.046 mol) of 1-(3-dimethylaminopropyl)-3-ethyl carbodiimide hydrochloride were added to the reaction mixture at room temperature and stirred for 15 hrs. DM water (30 ml) was added to the reaction mixture and aqueous layer was extracted with MDC (3×100 ml). Combined MDC layer was washed with brine solution (1×50 ml) and distilled under vacuum to get viscous liquid which was purified by column chromatography (silica gel 230-400 mesh, toluene:ethyl acetate, 30:70) to furnish 1-pyrrolidin-1-ylpropane-1,2-dione-2-oxime.

### Reference Example-III

### Preparation of (S)-3-[(2-hydroxyimino propionylamino)methyl]-5-methyl hexanoic acid

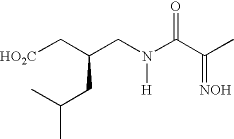

3.8 gm (0.027 mol) of potassium carbonate was added to a stirred heterogeneous solution of 4.36 gm (0.027 mol) of (S)-(+)-4-amino-3-(2-methylpropyl)butanoic acid in DMF (30 ml) and stirred for 30 minutes at room temperature. 3.0 gm (0.23 mol) of ethyl-2-hydroxyiminopropionate was added and the reaction mixture was heated for 7 hrs at 120° C. Reaction mixture was cooled to room temperature, concentrated under vacuum and DM water (25 ml) was added to the residue. Aqueous layer was acidified (pH˜4) with 2N HCl solution and extracted with ethyl acetate (3×50 ml). Combined ethyl acetate layer was washed with brine solution (1×30 ml) and concentrated under vacuum to get viscous liquid which was purified by column chromatography (silica gel 230-400 mesh, n-hexane:ethyl acetate, 30:70) to furnish (S)-3-[(2-hydroxyimino propionylamino)methyl]-5-methyl hexanoic acid.

The following examples illustrate the method of preparing certain representative compounds of formula-I.

### Example 5

### N-[(ethyl oximinopropionate-2-yl)carbonyl]-1-aminomethyl cyclohexaneacetic acid

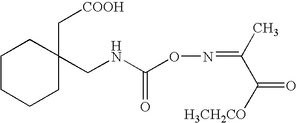

### Step-I: Preparation of [1-({[(4-nitrophenoxy)carbonyl]amino}methyl)cyclohexyl]acetic acid

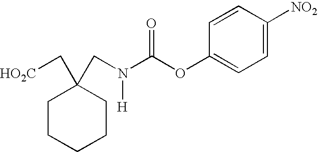

To a solution of 100 gm (0.584 mol) of gabapentine in 400 ml MDC was added 100 gm of triethylamine (0.99 mol) and cooled to 5-10° C. and 95.20 gm (0.876 mol) of trimethylchlorosilane was added between 5-10° C. and stirred for 45 minutes. To the reaction mixture added a solution of 107.2 gm (0.532 mol) of 4-nitrophenylchloroformate in 300 ml MDC at 0-5° C. and stirred the reaction at 20-25° C. for 3.0 hrs. To the reaction mixture added 700 ml DM water at below 10° C. The reaction mixture was extracted with MDC, the MDC layer was washed with 1N HCl solution and DM water followed by brine solution. The MDC layer was distilled and the degassed mass was dissolved in 280 ml toluene at 50-55° C. and added 120 ml n-hexane and stirred at room temperature for 3.0 hrs and the resultant solid was filtered and washed with a mixture of n-hexane and toluene and further washed with DM water, dried at 50-55° C. under vacuum to get [1-({[(4-nitrophenoxy)carbonyl]amino}methyl)cyclohexyl]acetic acid.

### Step-II: Preparation of N-[(ethyl oximinopropionate-2-yl)carbonyl]-1-aminomethyl cyclohexaneacetic acid

To a solution of 38.9 gm (0.297 mol) of ethyl-2-hydroxyiminopropionate in 500 ml MIBK was added 33.3 gm (0.297 mol) of potassium tert-butoxide at 0-5° C. and stirred at 25-30° C. for 30 minutes. Reaction mass was cooled to 0-5° C. and added 100 gm (0.297 mol) of [1-({[(4-nitrophenoxy)carbonyl]amino}methyl)cyclohexyl]acetic acid at 0-5° C. and stirred for 2.0 hrs at room temperature. To the reaction mixture added 400 ml of DM water followed by 220 ml 2N aqueous HCl at below 15° C. and extracted with MIBK and MIBK layer was washed with brine solution and distilled at 50-55° C. under vacuum. To the degassed mass added 2200 ml of n-hexane and 750 ml of ethylacetate and heated to get clear solution and cooled to 0-5° C. and stir for 2.0 hrs, filtered and washed with a mixture of chilled n-hexane and ethylacetate and further washed with DM water, dried at 50-55° C. under vacuum to get compound N-[(ethyl oximinopropionate-2-yl)carbonyl]-1-aminomethyl cyclohexaneacetic acid which was further purified with acetone and water mixture to get pure compound.

Compounds of examples 1-4 and 6-11 were prepared following the same procedure as example 5.

### Example 14

### N-[(oximinopropionic acid pyrrolidine amide-2-yl)carbonyl]-1-aminomethyl cyclohexaneacetic acid

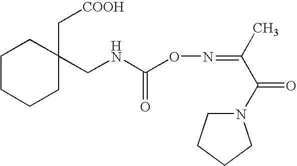

### Step 1: Preparation of [1-({[(4-nitrophenoxy)carbonyl]amino}methyl)cyclohexyl]acetic acid

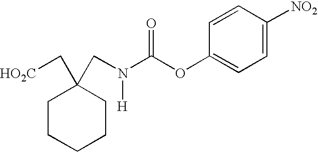

To a solution of 100 gm (0.584 mol) of gabapentine in 400 ml MDC was added 100 gm of triethylamine (0.99 mol) and cooled to 5-10° C. and 95.20 gm (0.876 mol) of trimethylchlorosilane was added between 5-10° C. and stirred for 45 minutes. To the reaction mixture added a solution of 107.2 gm (0.532 mol) of 4-nitrophenylchloroformate in 300 ml MDC at 0-5° C. and stirred the reaction at 20-25° C. for 3.0 hrs. To the reaction mixture added 700 ml DM water at below 10° C. The reaction mixture was extracted with MDC, the MDC layer was washed with 1N HCl solution and DM water followed by brine solution. The MDC layer was distilled and the degassed mass was dissolved in 280 ml toluene at 50-55° C. and added 120 ml n-hexane and stirred at room temperature for 3.0 hrs and the resultant solid was filtered and washed with a mixture of n-hexane and toluene and further washed with DM water, dried at 50-55° C. under vacuum to get [1-({[(4-nitrophenoxy)carbonyl]amino}methyl)cyclohexyl]acetic acid.

### Step II: Preparation of N-[(oximinopropionic acid pyrrolidine amide-2-yl)carbonyl]-1-aminomethyl cyclohexaneacetic acid

0.62 gm (0.006 mol) of potassium tert-butoxide was added to a stirred solution of 0.9 gm (0.006 mol) of 1-pyrrolidin-1-ylpropane-1,2-dione-2-oxime in methyl isobutyl ketone (20 ml) at 0-5° C. and then stirred for 30 minutes at 30° C. Reaction mixture was cooled to 0-5° C., 1.5 gm (0.004 mol) of N-[(4-nitrophenoxy)carbonyl]-1-aminomethyl cyclohexaneacetic acid was added to the reaction mixture at 0-5° C. and then stirred for 2 hrs at 30° C. DM water (30 ml) was added to the reaction mixture, organic layer was separated and aqueous layer was acidified (pH˜4) with 2N HCl solution. Aqueous layer was extracted with ethyl acetate (3×30 ml). Combined organic layer was washed with DM water (1×30 ml) followed by brine solution (1×30 ml) and distilled under vacuum to get viscous liquid which was purified by column chromatography (silica gel 230-400 mesh, ethyl acetate:methanol, 90:10) to furnish N-[(oximinopropionic acid pyrrolidine amide-2-yl)carbonyl]-1-aminomethyl cyclohexaneacetic acid.

Compounds of examples 12 and 13 were prepared in a manner analogous to compound 14.

### Example 15

### N-[(ethyl oximinopropionate-2-yl)carbonyl]-4-amino-3-(4-chloro phenyl)butanoic acid

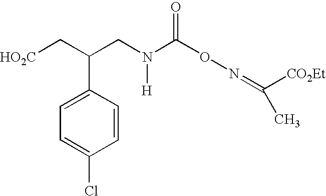

### Step-I: Preparation of 3-(4-chlorophenyl)-4-{[(4-nitrophenoxy)carbonyl]amino}butanoic acid

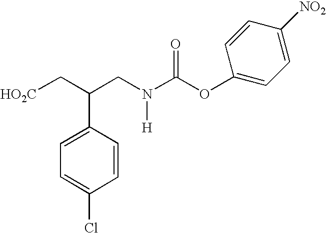

312.0 ml (2.458 mol) of trimethylchlorosilane was added slowly to the suspension of 350.0 gm (1.638 mol) of baclofen in THF (1400 ml) in presence of 387.0 ml (2.78 mol) of triethylamine at 0-5° C. over 1.5 hrs and maintained at 0-5° C. for 30 minutes. Solution of 347.0 gm (1.721 mol) of 4-nitrophenylchlorofomate in THF (1050 ml) was added to the above reaction mixture between 0-5° C. over 1.5 hrs and maintained at 25-30° C. for 2.5 hrs. Reaction mixture was cooled to below 10° C. and added DM water (1750 ml) followed by 1N HCl (1750 ml) and separated organic layer at room temperature. The aqueous layer was saturated with sodium chloride (600 gm) and extracted with THF (2×875 ml). Combined organic layer was dried over anhydrous sodium sulfate and solvent distilled under vacuum. Resulting solid was dissolved in IPA (1900 ml) at 75-80° C. and cooled to 0-5° C. and product was filtered, washed with chilled IPA (400 ml), suck dried and finally dried at 50-55° C. under vacuum to get 3-(4-chlorophenyl)-4-{[(4-nitrophenoxy)carbonyl]amino}butanoic acid.

### Step-II: Preparation of N-[(ethyl oximinopropionate-2-yl)carbonyl]-4-amino-3-(4-chloro phenyl)butanoic acid

142.3 gm (1.268 mol) of potassium tert-butoxide was added to the solution of 166.3 gm (1.268 mol) of ethyl-2-hydroxyiminopropionate in absolute ethanol at 0-5° C. over 25-30 minutes and stirred at 25-30° C. for 30 minutes. To the above solution 300.0 gm (0.792 mol) of 3-(4-chlorophenyl)-4-{[(4-nitrophenoxy)carbonyl]amino}butanoic acid was added over 25-30 minutes between 0-5° C. and stirred for 1.0 hr. Ethanol was distilled and degassed under vacuum, added DM water (3000 ml) and 2N HCl (550 ml). Aqueous layer was extracted with MDC (3×1500 ml) and combined organic layer was washed with DM water (1×1500 ml) followed by saturated brine solution (1×1500 ml). MDC was distilled and degassed under vacuum. Resulting degassed mass was dissolved in diisopropyl ether (1500 ml) and extracted with saturated sodium bicarbonate solution (3×600 ml). The combined aqueous layer pH was adjusted to ˜3.0 and extracted with MDC (3×1500 ml). Combined MDC layer was washed with DM water (1×1500 ml) followed by saturated brine Solution (1×1500 ml) and MDC was distilled. To the resulting thick mass hexane (930 ml) was added, heated at 65-70° C. and added ethyl acetate (620 ml) and gradually cooled to 20-25° C. Resulting solid was filtered, washed with mixture of ethyl acetate and n-hexane followed by DM water (3×350 ml) and dried at 50-55° C. under vacuum to get N-[(ethyl oximinopropionate-2-yl)carbonyl]-4-amino-3-(4-chlorophenyl)-butanoic acid which was further purified with methanol and water mixture to get pure product.

Compounds of example 16-21 were prepared following the same procedure as that of example 15.

### Example 23

### (±)-threo-N-[(ethyl oximinopropionate-2-yl)carbonyl]-1-phenyl-1-(2-piperidine)acetic acid methyl ester

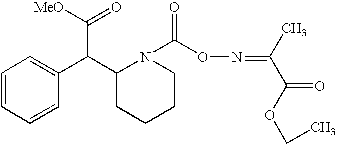

### Step-I: Preparation of (±)-threo-N-[(4-nitrophenoxy carbonyl]-1-phenyl-1-(2-piperidine)acetic acid methyl ester

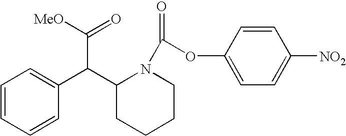

3.84 ml (0.022 mol) of N,N-diisopropylethylamine was added to a stirred solution of 4.0 gm (0.017 mol) of (±)-threo 1-phenyl-1-(2-piperidyl)acetic acid methyl ester in THF (40 ml) at 25-30° C. 4.14 gm (0.02 mol) of 4-nitrophenylchloroformate was added to the reaction mixture in portions over a period of 10 minutes and stirred at room temperature for 1 hr. Reaction mixture was concentrated under vacuum. DM water (40 ml) was added to the residue and extracted with MDC (3×40 ml). Combined MDC layer was washed with DM water (1×40 ml) followed by brine solution (1×40 ml). Finally MDC layer was distilled under vacuum to give yellow solid, which was crystallised from n-hexane ethylacetate (2:1) mixture (120 ml) to get (±)-threo-N-[(4-nitrophenoxy)carbonyl]-1-phenyl-1-(2-piperidine)acetic acid methyl ester.

### Step-II: Preparation of (±)-threo-N-[(ethyl oximinopropionate-2-yl)carbonyl]-1-phenyl-1-(2-piperidine)acetic acid methyl ester

0.47 gm (0.01 mol) of sodium hydride (˜50% suspension in oil) was added in portions to a stirred solution of 1.28 gm (0.01 mol) of ethyl-2-hydroxyiminopropionate in THF (20 ml) at 0-5° C. and stirred at room temperature for 30 minutes. A solution of 3.0 gm (0.007 mol) of (±)-threo-N-[(4-nitrophenoxy)carbonyl]-1-phenyl-1-(2-piperidine)acetic acid methyl ester in THF (10 ml) was added to the reaction mixture at 0-5° C. and stirred for 5 hrs at 25-30° C. Tetrahydrofuran was distilled and degassed under vacuum. DM water (30 ml) was added to the residue and extracted with MDC (3×30 ml). Combined MDC layer was washed with DM water (1×30 ml) followed by brine solution (1×30 ml). Finally MDC layer was distilled under vacuum to get viscous liquid which was purified by column chromatography (silica gel 230-400 mesh, n-hexane:ethyl acetate, 60:40) to furnish (±)-threo-N-[(ethyl oximinopropionate-2-yl)carbonyl]-1-phenyl-1-(2-piperidine)acetic acid methyl ester.

Compounds of example 22 were prepared following the same procedure as that of example 23.

### Example 24

### N-[(ethyl oximinopropionate-2-yl)carbonyl]-1,1-dimethyl-2-phenyl ethylamine

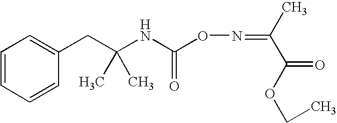

### Step-I: Preparation of N-[(4-nitrophenoxy)carbonyl]-1,1-dimethyl-2-phenyl ethylamine

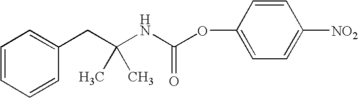

13.0 ml (0.081 mol) of N,N-diisopropylethylamine was added to a stirred solution of 11.0 gm (0.073 mol) of 1,1-dimethyl-2-phenyl ethylamine in THF (110 ml) at 25-30° C. 13.5 gm (0.02 mol) of 4-nitrophenylchloroformate was added to the reaction mixture in portions over a period of 20 minutes and stirred at room temperature for 5 hrs. Reaction mixture was concentrated under vacuum. DM water (100 ml) was added to the residue and extracted with MDC (3×100 ml). Combined MDC layer was washed with DM water (4×100 ml) followed by brine solution (4×100 ml) and distilled under vacuum to give viscous liquid. To which n-hexane (100 ml) was added and stirred for 10 minutes. Yellow solid thus obtained was filtered and washed with n-hexane (2×100 ml) followed by DM water (3×100 ml) to get N-[(4-nitrophenoxy)carbonyl]-1,1-dimethyl-2-phenyl ethylamine.

### Step-II: Preparation of N-[(ethyl oximinopropionate-2-yl)carbonyl]-1,1-dimethyl-2-phenyl ethylamine

0.62 gm (0.013 mol) of sodium hydride (˜50% suspension in oil) was added in portions to a stirred solution of 1.7 gm (0.013 mol) of ethyl-2-hydroxyiminopropionate in THF (10 ml) at 0-5° C. and stirred at room temperature for 30 minutes. A solution of 3.0 gm (0.01 mol) of N-[(4-nitrophenoxy)carbonyl]-1,1-dimethyl-2-phenyl ethylamine in THF (20 ml) was added to the reaction mixture at 0-5° C. and stirred at 25-30° C. for 2 hrs. Tetrahydrofuran was distilled and degassed under vacuum. DM water (45 ml) was added to the residue and extracted with MDC (3×30 ml). Combined MDC layer was washed with DM water (1×30 ml) followed by brine solution (1×30 ml) and distilled under vacuum to get viscous liquid which was purified by column chromatography (silica gel 230-400 mesh, n-hexane:ethyl acetate, 85:15) to furnish N-[(ethyl oximinopropionate-2-yl)carbonyl]-1,1-dimethyl-2-phenyl ethylamine.

### Example 25

### (S)-N-[(ethyl oximinopropionate-2-yl)carbonyl]-4-amino-3-(2-methylpropyl)butanoic acid

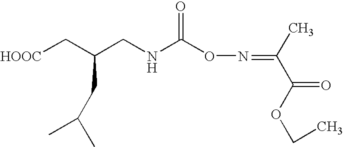

### Step-I: Preparation of (S)-N-[(4-nitrophenoxy)carbonyl]-4-amino-3-(2-methylpropyl)butanoic acid

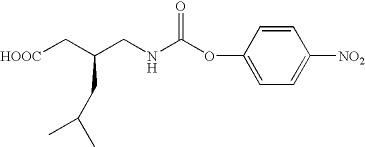

38.5 ml (0.276 mol) of triethylamine was added to a stirred solution of 20.0 gm (0.125 mol) of (S)-(+)-4-amino-3-(2-methylpropyl)butanoic acid in MDC (100 ml) and cooled to 5-10° C. 23.9 ml (0.188 mol) of trimethylchlorosilane was added slowly to the reaction mixture at 5-10° C. and stirred for 30 minutes. A solution of 25.3 gm (0.125 mol) of 4-nitrophenylchloroformate in MDC (100 ml) was added slowly to the reaction mixture at 5-10° C. and stirred for 4 hrs at room temperature. DM water (200 ml) was added to the reaction mixture at 5-10° C., organic layer was separated and aqueous layer was extracted with MDC (2×100 ml). Combined MDC layer was washed with 2N HCl solution (1×200 ml) followed by DM water (1×200 ml) and brine solution (1×200 ml). MDC layer was distilled and degassed under vacuum to get viscous liquid. n-Hexane-toluene (80:20) mixture (420 ml) was added to the viscous liquid and stirred for 3 hrs. Light yellow solid thus obtained was filtered and washed with n-hexane-toluene (80:20) mixture (2×210 ml) followed by n-hexane (2×210 ml) to furnish (S)-N-[(4-nitrophenoxy)carbonyl]-4-amino-3-(2-methylpropyl)butanoic acid.

### Step-II: Preparation of (S)-N-[(ethyl oximinopropionate-2-yl)carbonyl]-4-amino-3-(2-methylpropyl)butanoic acid

8.65 gm (0.077 mol) of potassium tert-butoxide was added to a stirred solution of 10.5 gm (0.08 mol) of ethyl-2-hydroxyiminopropionate in methyl-isobutyl ketone (200 ml) at 0-5° C. and then stirred for 30 minutes at 25-30° C. Reaction mixture was cooled to 0-5° C. 20.0 gm (0.061 mol) of (S)-N-[(4-nitrophenoxy)carbonyl]-4-amino-3-(2-methylpropyl)butanoic acid was added to the reaction mixture at 0-5° C. and then stirred for 1 hr at 25-30° C. DM water (200 ml) was added to the reaction mixture and organic layer was separated. Aqueous layer was acidified (pH˜4) with 2N HCl solution and extracted with ethyl acetate (2×200 ml). Combined organic layer was washed with brine solution (1×200 ml) and concentrated under vacuum to get viscous liquid which was purified by column chromatography (silica gel 230-400 mesh, n-hexane:ethyl acetate, 40:60) to furnish (S)-N-[(ethyl oximinopropionate-2-yl)carbonyl]-4-amino-3-(2-methylpropyl)butanoic acid.

### Example 26

### trans-N-[(ethyl oximinopropionate-2-yl)carbonyl]-4-(aminomethyl)cyclohexanecarboxylic acid

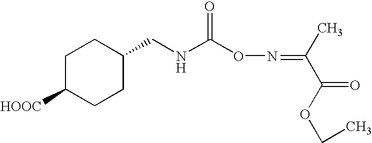

### Step-I: Preparation of trans-N-[(4-nitrophenoxy)carbonyl]-4-(aminomethyl)cyclohexane carboxylic acid

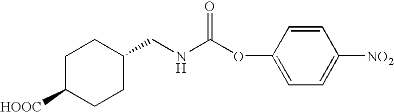

22.6 ml (0.162 mol) of triethylamine was added to a stirred solution of 15.0 gm (0.095 mol) of trans-(4-aminomethyl)cyclohexanecarboxylic acid in MDC (75 ml) and cooled to 5-10° C. 17.3 ml (0.143 mol) of trimethylchlorosilane was added slowly to the reaction mixture at 5-10° C. and stirred for 30 minutes. A solution of 20.2 gm (0.1 mol) of 4-nitrophenylchloroformate in MDC (45 ml) was slowly added to the reaction mixture at 5-10° C. and stirred for 4 hrs at room temperature. DM water (75 ml) followed by 2N HCl solution were added slowly to the reaction mixture at 5-10° C. White solid thus obtained was filtered and washed with DM water (3×50 ml) followed by MDC (2×50 ml) to furnish trans-N-[(4-nitrophenoxy)carbonyl]-4-(aminomethyl)cyclohexanecarboxylic acid.

### Step-II: Preparation of trans-N-[(ethyl oximinopropionate-2-yl)carbonyl]-4-(aminomethyl)cyclohexanecarboxylic acid

6.68 gm (0.059 mol) of potassium tert-butoxide was added to a stirred solution of 7.73 gm (0.059 mol) of ethyl-2-hydroxyiminopropionate in THF (190 ml) at 0-5° C. and then stirred for 30 minutes at 25-30° C. Reaction mixture was cooled to 0-5° C. 19.0 gm (0.059 mol) of trans-N-[(4-nitrophenoxy)carbonyl]-4-(aminomethyl)cyclohexanecarboxylic acid was added to the reaction mixture at 0-5° C. and then stirred for 4 hrs at 25-30° C. Tetrahydrofuran was distilled and degassed under vacuum at 35° C. DM water (100 ml) was added to the residue, aqueous layer was acidified (pH˜4) with 2N HCl solution and extracted with MDC (3×100 ml). Combined MDC layer was washed with DM water (1×100 ml) followed by brine solution (1×100 ml) and concentrated under vacuum to get viscous liquid which was purified by column chromatography (silica gel 230-400 mesh, toluene:ethyl acetate, 50:50) to furnish trans-N-[(ethyl oximinopropionate-2-yl)carbonyl]-4-(aminomethyl)cyclohexanecarboxylic acid.

Compounds of example 27-29 were prepared following a similar procedure as that of example 26

The following compounds may be prepared in a manner similar to compounds of examples 1-29 as disclosed above.

| Example No. | Compound |
|---|---|
| 30 | 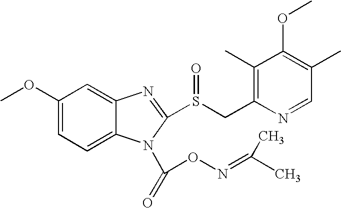 |
| 31 | 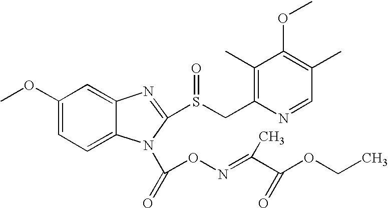 |
| 32 | 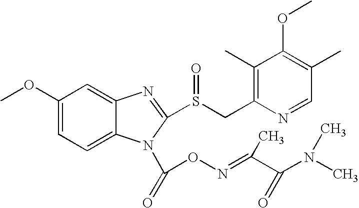 |
| 33 | 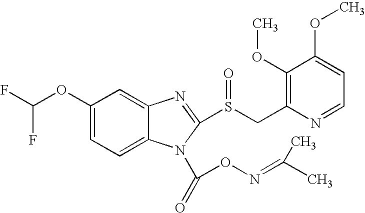 |
| 34 | 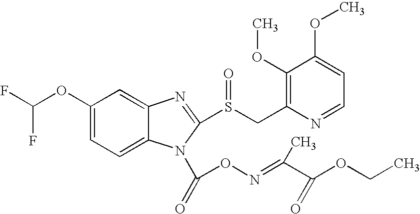 |
| 35 | 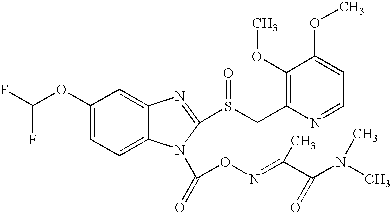 |
| 36 | 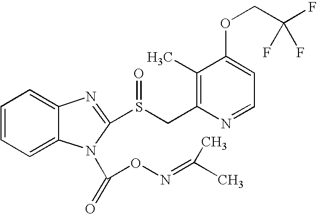 |
| 37 | 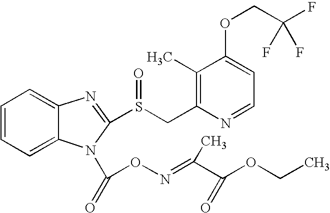 |
| 38 | 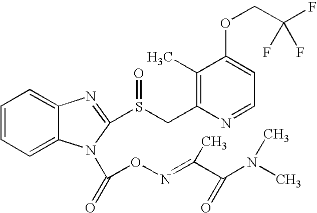 |
| 39 | 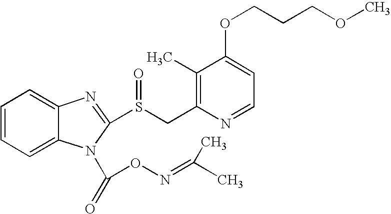 |
| 40 | 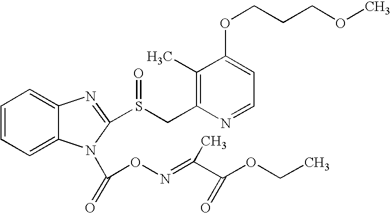 |
| 41 | 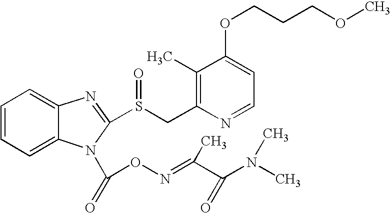 |

Table 1 illustrates the chemical structures and the mass spectrometry data of the representative examples.

| Example No | Compound | MS |
|---|---|---|
| 1 | 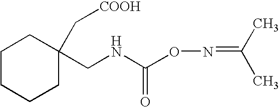 | 293.26 (M + Na)⁺ |
| 2 | 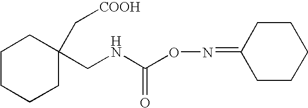 | 333.2 (M + Na)⁺ |
| 3 | 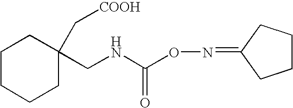 | 319.22 (M + Na)⁺ |
| 4 | 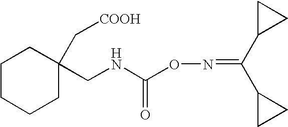 | 345.21 (M + Na)⁺ |
| 5 | 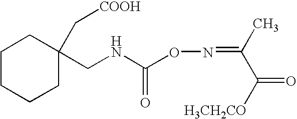 | 351.1 (M + Na)⁺ |
| 6 | 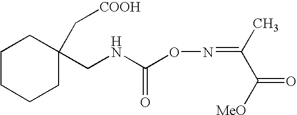 | 337.21 (M + Na)⁺ |
| 7 | 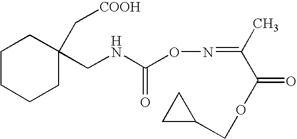 | NA* |
| 8 | 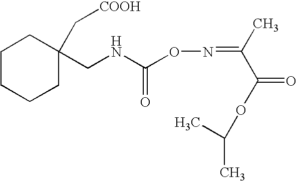 | 365.22 (M + Na)⁺ |
| 9 | 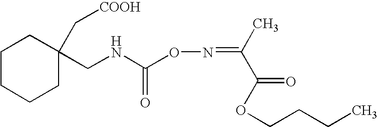 | 379.20 (M + Na)⁺ |
| 10 | 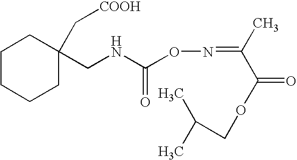 | 379.20 (M + Na)⁺ |
| 11 | 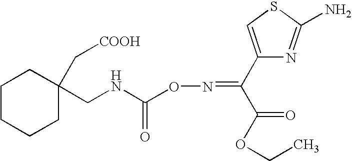 | 435.18 (M + Na)⁺ |
| 12 | 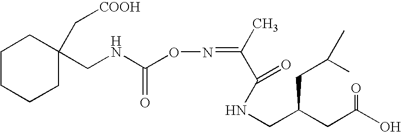 | 442.20 (M + H)⁺ |
| 13 | 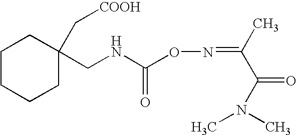 | 350.17 (M + Na)⁺ |
| 14 | 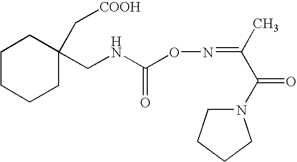 | 376.13 (M + Na)⁺ |
| 15 | 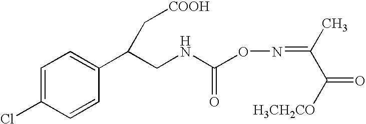 | 392.94 (M + Na)⁺ |
| 16 | 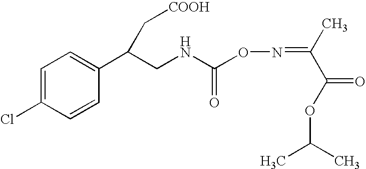 | 406.96 (M + Na)⁺ |
| 17 | 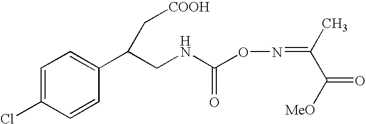 | 378.93 (M + Na)⁺ |
| 18 | 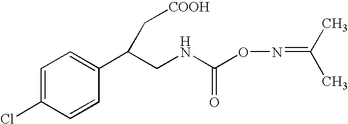 | 335.0 (M + Na)⁺ |
| 19 | 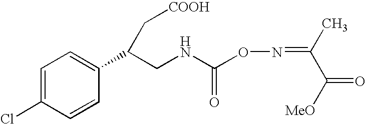 | 357.01 (M + H)⁺ |
| 20 | 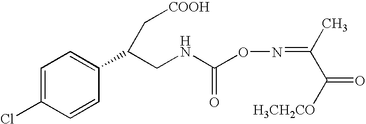 | 393.08 (M + Na)⁺ |
| 21 | 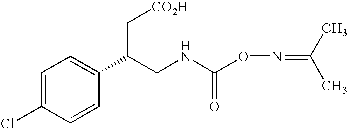 | 335.08 (M + Na)⁺ |
| 22 | 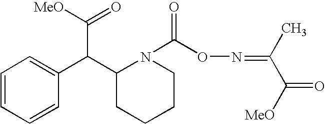 | 377.22 (M + H)⁺ |
| 23 | 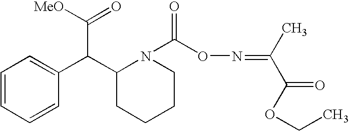 | 391.23 (M + H)⁺ |
| 24 | 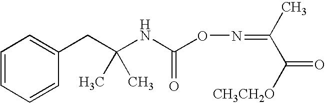 | 329.20 (M + H)⁺ |
| 25 | 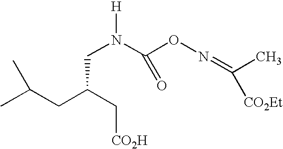 | 339.21 (M + Na)⁺ |
| 26 | 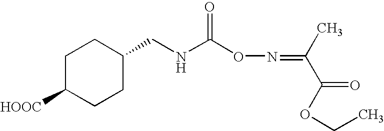 | 337.12 (M + Na)⁺ |
| 27 | 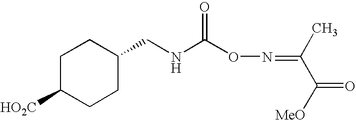 | 323.15 (M + Na)⁺ |
| 28 | 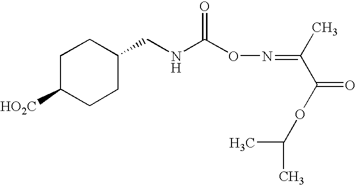 | 351.17 (M + Na)⁺ |
| 29 | 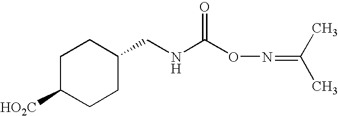 | 279.15 (M + Na)⁺ |

*Not available

### Conversion of the Compounds of the Present Invention to the Active Compounds

The prodrugs compounds of the present invention, i.e. the compounds of formula-I release in-vivo the compounds of formula III. Two of the representative compounds of the present invention i.e. compound of example 5 and compound 15, were tested to determine the comparative bioavailability of the drug and the prodrug. The following method was employed for the determination of bioavailability of the test compound of example 5.

Liquid Chromatography Mass Spectrometric Method for the Determination of Gabapentin from Example 5 in Plasma.

Mobile phase: Prepare 2 mM ammonium acetate solution in milliQ water and adjust the pH to 3.0 with formic acid. Mix above buffer solution and acetonitrile in the ratio (30:70 v/v) and filter.

### Chromatographic Conditions:

Column: Hypurity C18, 50×2.1 mm, 5μ; Column oven temperature: 40° C.; Flow rate: 0.25 ml/min; Injection volume: 10 μl; Run time: 2.0 min.; Retention time: Example 5: 1.0 min.; Gabapentin: 1.0 min.; Carbamazepine: 1.0 min.;

Mass Parameters: Spray Voltage: 3500 V; Sheath Gas Pressure: 35 ml/min; Capillary Temperature: 380° C.; Mode: Positive; Aux gas pressure: 10 ml/min

| | | |
|---|---|---|
| Example 5 | Parent mass - 329.120 | Product mass - 85.972, 154.044 |
| Gabapentin | Parent mass - 172.100 | Product mass - 137.034, 154.054 |
| Carbamazepine | Parent mass - 237.058 | Product mass - 194.003 |
| (IS) | | |

### Preparation of Standard Solutions:

For Gabapentin Linearity Range: 200 ng-19600 ng/ml in mobile phase.

For example 5: Linearity Range: 50 ng 5000 ng/ml in mobile phase.

### Sample Preparation:

A 100 μl plasma sample and 5 μl of internal standard was taken in a micro centrifuge tube. Vortex for 20-30 sec. The test samples were loaded in preconditioned HLB cartridges. The cartridge was washed with 1 ml Milli-Q water the sample was eluted with 500 μl of mobile phase. The sample was centrifuged at 15000 rpm for 5 minutes and the supernatant was collected for analysis.

Table 1 and Table 2 below provide Dose-concentration data for the representative compounds 5 and compound 15 of the invention, which are prodrugs of gabapentin and baclofen respectively.

TABLE 1

| | |
|---|---|
| Gabapentin | Compound of example 5 |

| Dose | AUC_{(0-t*)} | Equivalent | AUC_{(0-t*)} |
|---|---|---|---|
| mg/kg, | mcg · hr/mL | Dose | mcg · hr/mL |
| p.o | (gabapentin) | ~mg/kg, p.o | (gabapentin) |
| 50 mg | 85 | 50 mg | 89 |
| 100 mg | 99 | 100 mg | 187 |

*t = 24 hrs

TABLE 2

| Baclofen | | | Compound of example 15 | | |
|---|---|---|---|---|---|
| Dose | | AUC₍₀₋ₜ*₎ | Equivalent | | AUC₍₀₋ₜ*₎ |
| mcg/kg, | Cmax | mcg · hr/mL | Dose | Cmax | mcg · hr/mL |
| p.o | mcg/mL | (Baclofen) | mcg/kg, p.o | mcg/mL | (Baclofen) |
| 20 | 3.6 | 15 | 20 | 8 | 24 |

*t = 24 hrs

As can be observed from the data in Table I, the gabapentin prodrug of the present invention provides higher bioavailability of gabapentin at the higher dose. It also provides for the dose-proportional bioavailability, whereas gabapentin, shows non-linear saturable absorption, with lower bioavailability at the higher dose. Similarly, it was found that the baclofen prodrug of the present invention provides higher bioavailability of baclofen as evidenced from the data in Table 2.

1-15. (canceled)

16. A compound of formula-I, or a pharmaceutically acceptable salt thereof: 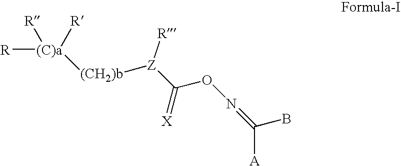
wherein the groups R, R′, R″ and R′″ are independently selected from hydrogen, linear alkyl, branched alkyl, cyclic alkyl, alkylaryl, aralkyl, aryl, or heterocyclic ring; wherein the alkyl group is independently saturated or unsaturated, and is independently optionally substituted with 1 to 5 groups selected from hydroxy, cyano, oxo, carboxylic acid, phosphoric acid, sulfonic acid, alkyl sulfonate, aryl sulfonate, aralkyl sulfonate, alkylaryl sulfonate, phosphoric acid, alkyl phosphonate, aryl phosphonate, aralkyl phosphonate, alkyl aryl phosphonate, CONR₁₂R₁₃ wherein R₁₂ and R₁₃ are independently selected from hydrogen, alkyl, or aryl; wherein the aryl group is unsubstituted or substituted with one or more alkyl; COOR_{z}, wherein R_{z} is selected from hydrogen, alkyl, aryl, alkylaryl, aralkyl, a cyclic ring, or a heterocyclic ring; wherein each aryl and heterocyclic ring is independently optionally substituted with 1 to 5 groups selected from alkyl, alkoxy, halo, perhaloalkyl, perhaloalkoxy, haloalkoxy, hydroxy, oxo, cyano, carboxy, acyl, —NR_{P}R_{Q}, wherein R_{P} and R_{Q} are independently selected from hydrogen, alkyl, arylalkyl, alkylaryl, cyclic or heterocyclic ring, and —CONR_{M}R_{N}, wherein R_{M} and R_{N} are each independently selected from hydrogen, alkyl, or aryl; wherein each aryl of R_{M} and R_{N} is optionally substituted with alkyl;
or any two of R, R′, R″ or R′″ are joined together to form a cyclic moiety which is optionally substituted with alkyl, perhaloalkyl, alkoxy, halogen, amino, alkylamino, dialkylamino, cyano, carboxy, alkoxycarbonyl, alkanoyl or a group L, wherein L is a compound of formula-P: 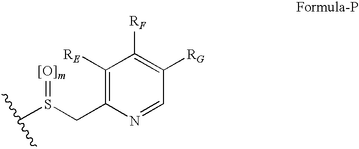
wherein m is 0 or 1; R_{E}, R_{F} and R_{G} are selected from one of the following groups:
i) R_{F} represents a C₁ to C₃ alkoxy group, one of the groups R_{E} and R_{G} represents a C₁ to C₃ alkoxy group and the other represents a hydrogen atom and a C₁ to C₃ alkyl radical; or
ii) R_{E} and R_{G} represents hydrogen or methyl; R_{F} represents a group of the formula OCH₂R_{I}, wherein R_{I} represents a fluorinated alkyl radical; or
iii) R_{E} and R_{G} are independently represent hydrogen, methyl, methoxy, ethoxy, methoxyethoxy or ethoxyethoxy; and R_{F} is selected from methoxy, ethoxy, methoxyethoxy or ethoxyethoxy; or
iv) R_{G} is hydrogen, R_{E} represents methyl, and R_{F} represents methoxy substituted by —O-n-propyl;
Z is an atom selected from N, O or S;
X is an atom selected from O or S;
A is selected from hydrogen, C₁ to C₁₀ linear alkyl, C₁ to C₁₀ linear branched alkyl, C₃ to C₇ cyclic alkyl, aryl or heterocyclic ring; wherein the alkyl group is completely saturated or contains unsaturation, and is either unsubstituted or substituted with 1 to 5 groups selected from hydroxy, halogen, cyano, carboxy, and acyl, wherein the aryl and the heterocyclic ring are each independently unsubstituted or substituted with 1 to 5 groups selected from alkyl, alkoxy, halo, perhaloalkyl, perhaloalkoxy, haloalkoxy, hydroxy, cyano, amino, monoalkylamino and dialkylamino groups;
B is selected from hydrogen, cyano, C₁ to C₁₀ alkyl, a group of the formula —COORa, wherein Ra is selected from hydrogen, C₁₋₁₀alkyl, aryl or heteroaryl moiety; or a group of the formula —CONRₓR_{y}, wherein Rₓ and R_{y} are independently selected from hydrogen, C₁ to C₇ linear alkyl, C₁ to C₇ branched alkyl, C₃ to C₇ cyclic alkyl, aryl or heterocyclic ring, wherein the alkyl group is completely saturated or contains unsaturation and is unsubstituted or substituted with 1 to 5 groups selected from hydroxy, halogen, cyano, carboxy, and acyl;
or B is a group of formula-II: 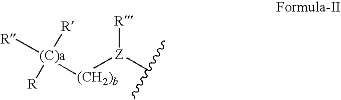
wherein R, R′, R″, R′″, Z have the meanings as defined above;
a is selected from 0 or 1;
b is selected from 0 or 1;
with a proviso that,
i) when a is 0, b is 0; R′ and R″ are absent and R is directly attached to Z;
ii) when Z is an atom selected from O or S; R′″ is absent;
iii) the compound of formula-I is converted to a compound of formula-III: 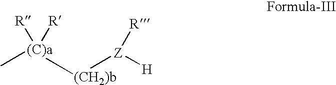
wherein R, R′, R″, R′″, a, b and Z are as defined above;
iv) the compound of formula-III is a biologically active molecule or is a diagnostic agent.

17. A compound according to claim 16, wherein the compound of formula-I is represented by a compound of formula-IV: 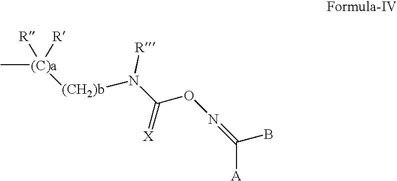
wherein, at least one of the groups R, R′ and R″ contains a carboxylic moiety and the other R, R′ and R″ groups have the meaning as defined in claim 1; and a, b, X, A and B have the meaning as defined in claim 16.

18. A compound according to claim 17, wherein the compound of formula-IV is represented by a compound of formula-V: 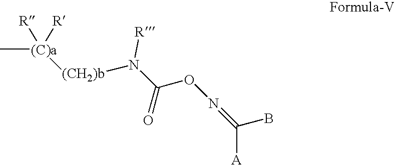
wherein, at least one of the groups R, R′ and R″ contains a carboxylic moiety and the other R, R′ and R″ groups have the meaning as defined in claim 16; and a, b, A and B have the meaning as defined in claim 16.

19. A compound according to claim 16, wherein the compound of formula-I is represented by a compound of formula-Ia: 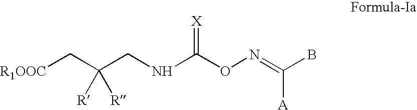
wherein R′ and R″ are connected together with the carbon atom to which they are attached to form a 4, 5 or 6-membered cyclic ring; R₁ is selected from hydrogen or a C₁-C₈ alkyl radical; and X, A and B have the meanings as defined in claim 16.

20. A compound as claimed in claim 16, wherein the compound of formula-I is represented by a compound of formula-Ib: 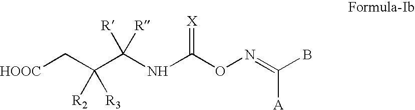
wherein R′ is hydrogen, R₂ is a straight or branched alkyl of from 1 to 6 carbons, phenyl or cycloalkyl having from 3 to 6 carbon atoms; R″ and R₃ are independently selected from hydrogen or methyl; and X, A and B have the meanings as defined in claim 16.

21. A compound as claimed in claim 16, wherein, the compound of formula-I is represented by a compound of formula-Ic: 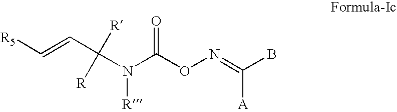
wherein R′ is hydrogen and R′″ is hydrogen;
R₅ is selected from hydrogen or chlorine;
R is a group of the formula —CH═CH—COR or —[CH(R₇)]ₙ—COR₆, wherein R₆ is selected from hydroxy, a straight or branched alkoxy group of from 1 to 8 carbon atoms, a lower alkylamino group; R₇ is selected from hydrogen, C₁ to C₄ alkyl, phenyl and substituted phenyl wherein the substituents on phenyl are selected from halogen, C₁ to C₄ alkoxy of from 1 to 4 carbon atoms, and C₁ to C₄ alkyl; n is an integer of from 1 to 5; and X, A and B have the meanings as defined in claim 16.

22. A compound as claimed in claim 16, wherein the compound of formula-I is represented by a compound of formula-Id: 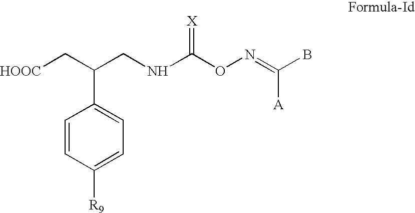
wherein R₉ is selected from a chlorine, bromine, iodine, —CF₃; and X, A and B have the meanings as defined in claim 16.

23. A compound as claimed in claim 16, wherein the compound of formula-I is represented by a compound of formula-Ie: 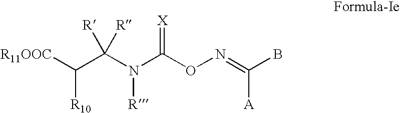
wherein R′ and R′″ are connected to form, together with the N atom to which they are attached, a piperidyl ring; R″ is H, R₁₀ is phenyl optionally substituted with C₁₋₄ alkyl; R₁₁ is C₁ to C₄ alkyl; X, and A and B have the meanings as defined in claim 16.

24. A compound as claimed in claim 16, wherein the compound of formula-I is represented by a compound of formula-If: 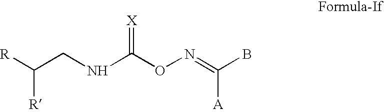
wherein R and R′ are connected together to form a 6-membered saturated cyclic ring, which is substituted by —COOH; and X, A and B have the meanings as defined in claim 16 above.

25. A compound as claimed in claim 16, wherein the compound of formula-I is represented by a compound of formula-Ig: 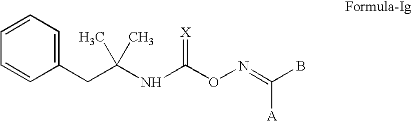
wherein the substitutents X, B and A have the meanings as defined in claim 16.

26. A compound as claimed in claim 16, wherein the compound of formula-I is represented by a compound of formula-Ih: 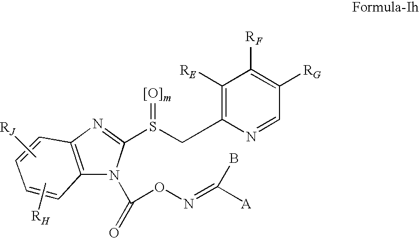
wherein m is 0 or 1; the groups R_{J}, R_{H}, R_{E}, R_{F} and R_{G} are selected from one of the following groups:
i) R_{F} represents a C₁ to C₃ alkoxy group, one of the groups; R_{E} and R_{G} represents a C₁ to C₃ alkoxy group and the other represents a hydrogen atom and a C₁ to C₃ alkyl radical; R_{J} is at the 6-position and represents hydrogen, halo, trifluoromethyl, a C₁ to C₃ alkyl radical or a C₁ to C₃ alkoxy radical which is optionally, predominantly or completely substituted by fluorine atoms; R_{H} is at the 5-position and represents a C₁ to C₃ alkoxy radical which is optionally, predominantly or completely substituted by fluorine atoms or a chlorodifluormethyl radical;
ii) R_{E} and R_{G} independently represent hydrogen or methyl; R_{F} represents a group of the formula —OCH₂R₁ wherein R₁ represents a fluorinated alkyl radical; R_{J} is hydrogen; R_{H} is selected from hydrogen, methoxy or trifluoromethyl;
iii) R_{E} and R_{G} independently represent hydrogen, methyl, methoxy, ethoxy, methoxyethoxy or ethoxyethoxy; R_{F} is selected from methoxy, ethoxy, methoxyethoxy or ethoxyethoxy; R_{J} and R_{H} are independently selected from the group consisting of hydrogen, alkyl, halogen, carbomethoxy, carboethoxy, alkoxy, and alkanoyl;
iv) R_{G} is hydrogen; R_{E} represents methyl; and R_{F} represents methoxy substituted by an —O-n-propyl; R_{J} and R_{H} are independently selected from the group consisting of hydrogen, halogen, C₁ to C₆ alkyl, halogenated C₁ to C₆ alkyl, C₁ to C₆ alkoxy, C₁ to C₆ alkoxycarbonyl or carboxyl group; and X, A and B have the meaning as defined in claim 16.

27. A compound as claimed in claim 16, wherein the compound of formula-I is represented by a compound of formula-Ii: 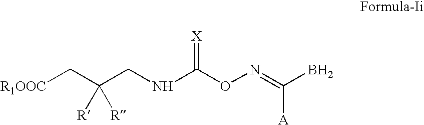
wherein R′ and R″ are connected together with the carbon atom to which they are attached to form a 4, 5 or 6-membered saturated cyclic ring; R₁ is selected from hydrogen atom or a C₁-C₈ alkyl radical;
B is a group of the formula-VIII: 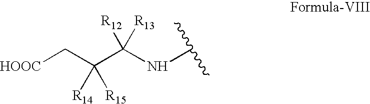
wherein R₁₂ is hydrogen; R₁₃ and R₁₅ are independently selected from hydrogen or methyl; R₁₄ is a straight or branched alkyl of from 1 to 6 carbons, phenyl or cycloalkyl having from 3 to 6 carbon atoms; and X and A have the meanings as defined in claim 16.

28. A compound according to claim 19, wherein the compound of formula-Ia is represented by a compound of formula Ij: 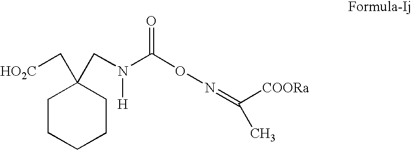
wherein Ra has the meaning as defined in claim 16.

29. A compound according to claim 22, wherein the compound of formula-Id is represented by a compound of formula-Ik: 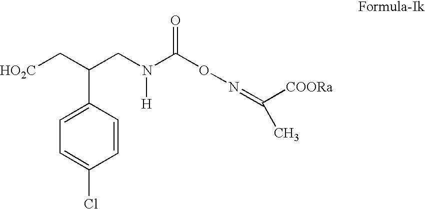
wherein Ra has the meaning as defined in claim 16.

30. A compound according to claim 16, selected from the group consisting of:
N-[(Oximinopropane-2-yl)carbonyl]-1-aminomethyl cyclohexaneacetic acid,
N-[(Oximinocyclohexane)carbonyl]-1-aminomethyl cyclohexaneacetic acid,
N-[(Oximinocyclopentane)carbonyl]-1-aminomethy cyclohexaneacetic acid,
N-[(Oximino-1,1-dicyclopropyl methane)carbonyl]-1-aminomethyl cyclohexaneacetic acid,
N-[(Ethyl oximinopropionate-2-yl)carbonyl]-1-aminomethyl cyclohexaneacetic acid,
N-[(Methyl oximinopropionate-2-yl)carbonyl]-1-aminomethyl cyclohexaneacetic acid,
N-[(Cyclopropyl methyl oximinopropionate-2-yl)carbonyl)-1-aminomethyl cyclohexaneacetic acid,
N-[(Isopropyl oximinopropionate-2-yl)carbonyl]-1-aminomethyl cyclohexaneacetic acid,
N-[(n-Butyl oximinopropionate-2-yl)carbonyl]-1-aminomethyl cyclohexaneacetic acid,
N-[(Isobutyl oximinopropionate-2-yl)carbonyl]-1-aminomethyl cyclohexaneacetic acid
N-[(Ethyl-2-{2-aminothiazole}oximinoethanoate-2-yl)carbonyl]-1-aminomethyl cyclohexaneacetic acid,
N-[(Oximinopropionic acid-{4-amino-3-(2-methylpropyl)butanoic}amide-2-yl)carbonyl]-1-aminomethyl cyclohexaneacetic acid,
N-[(Oximinopropionic acid dimethyl amide-2-yl)carbonyl]-1-aminomethyl cyclohexaneacetic acid,
N-[(Oximinopropionic acid pyrrolidine amide-2-yl)carbonyl]-1-aminomethyl cyclohexaneacetic acid,
N-[(Ethyl oximinopropionate-2-yl)carbonyl]-4-amino-3-(4-chlorophenyl)butanoic acid,
N-[(Isopropyl oximinopropionate-2-yl)carbonyl]-4-amino-3-(4-chlorophenyl)butanoic acid,
N-[(Methyl oximinopropionate-2-yl)carbonyl]-4-amino-3-(4-chlorophenyl)butanoic acid,
N-[(Oximinopropane-2-yl)carbonyl]-4-amino-3-(4-chlorophenyl)butanoic acid,
(R)-N-[(Methyl oximinopropionate-2-yl)carbonyl]-4-amino-3-(4-chlorophenyl)butanoic acid,
(R)-N-[(Ethyl oximinopropionate-2-yl)carbonyl]-4-amino-3-(4-chlorophenyl)butanoic acid,
(R)-N-[(Oximinopropane-2-yl)carbonyl]-4-amino-3-(4-chlorophenyl)butanoic acid,
(±)-Threo-N-[(Methyl oximinopropionate-2-yl)carbonyl]-1-phenyl-1-(2-piperidine)acetic acid methyl ester,
(±)-Threo-N-[(Ethyl oximinopropionate-2-yl)carbonyl]-1-phenyl-1-(2-piperidine)acetic acid methyl ester,
N-[(Ethyl oximinopropionate-2-yl)carbonyl]-1,1-dimethyl-2-phenyl ethylamine,
(S)-N-[(Ethyl oximinopropionate-2-yl)carbonyl]-4-amino-3-(2-methylpropyl)butanoic acid,
Trans-N-[(Ethyl oximinopropionate-2-yl)carbonyl]-4-(aminomethyl)cyclohexanecarboxylic acid,
Trans-N-[(Methyl oximinopropionate-2-yl)carbonyl]-4-(aminomethyl)cyclohexanecarboxylic acid,
Trans-N-[(Isopropyl oximinopropionate-2-yl)carbonyl]-4-(aminomethyl)cyclohexanecarboxylic acid,
Trans-N-[(Oximinopropane-2-yl)carbonyl]-4-(aminomethyl)cyclohexane carboxylic acid,
N-[(Oximinopropane-2-yl)carbonyl]-5-methoxy-2-([(4-methoxy-3,5-dimethyl-2-pyridinyl]methyl]sulfinyl]-1H-benzimidazole,
N-[(Ethyl oximinopropionate-2-yl)carbonyl]-5-methoxy-2-[[(4-methoxy-3,5-dimethyl-2-pyridinyl)methyl]sulfinyl]-1H-benzimidazole,
N-[(Oximinopropionic acid dimethyl amide-2-yl)camonyl]-5-methoxy-2-[[(4-methoxy-3,5-dimethyl-2-pyridinyl)methyl]sulfinyl]-1H-benzimidazole,
N-[(Oximinopropane-2-yl)carbonyl]-5-(difluoromethoxy)-2-[[(3,4-dimethoxy-2-pyridinyl)methyl]sulfinyl]-1H-benzimidazole,
N-[(Ethyl oximinopropionate-2-yl)carbonyl]-5-(difluoromethoxy)-2-[[(3,4-dimethoxy-2-pyridinyl)methyl]sulfinyl]-1H-benzimidazole,
N-[(Oximinopropionic acid dimethyl amide-2-yl)carbonyl]-5-(difluoromethoxy)-2-[[(3,4-dimethoxy-2-pyridinyl)methyl]sulfinyl]-1H-benzimidazole,
N-[(Oximinopropane-2-yl)carbonyl]-2-[[[3-methyl-4-(2,2,2-trifluoroethoxy)-2-pyridinyl]methyl]sulfinyl]-1H-benzimidazole,
N-[(Ethyl oximinopropionate-2-yl)carbonyl]-2-[[[3-methyl-4-(2,2,2-trifluoroethoxy)-2-pyridinyl]methyl]sulfinyl]-1H-benzimidazole,
N-[(Oximinopropionic acid dimethyl amide-2-yl)carbonyl]-2-[[[3-methyl-4-(2,2,2-trifluoroethoxy)-2-pyridinyl]methyl]sulfinyl]-1H-benzimidazole,
N-[(Oximinopropane-2-yl)carbonyl]-2-[[[4-(3-methoxypropoxy)-3-methyl-2-pyridinyl]methyl]sulfinyl]-1H-benzimidazole,
N-[(Ethyl oximinopropionate-2-yl)carbonyl]-2-[[[4-(3-methoxypropoxy)-3-methyl-2-pyridinyl]methyl]sulfinyl]-1H-benzimidazole, and
N-[(Oximinopropionic acid dimethyl amide-2-yl)carbonyl]-2-[[[4-(3-methoxypropoxy)-3-methyl-2-pyridinyl]methyl]sulfinyl J-1H-benzimidazole.
